(19) United States
(12) Patent Application Publication
(10) Pub. No.: US 2025/0248915 A1
(43) Pub. Date: Aug. 7, 2025

(54) COMPOSITIONS AND METHODS FOR TREATING HAIR

(71) Applicant: L'Oréal, Paris (FR)

(72) Inventors: Jennifer ELIE, Piscataway, NJ (US); Marc ROSENBERG, Westfield, NJ (US)

(21) Appl. No.: 19/041,130

(22) Filed: Jan. 30, 2025

Related U.S. Application Data
(60) Provisional application No. 63/548,778, filed on Feb. 1, 2024.

(30) Foreign Application Priority Data
Apr. 29, 2024 (FR) 2404457

(51) Int. Cl. A61K 8/44; A61K 8/365; A61Q 5/00

(52) U.S. Cl. CPC A61K 8/44; A61K 8/365; A61Q 5/002; A61Q 5/004; A61K 2800/4322; A61K 2800/4324; A61K 2800/882

(57) ABSTRACT

The disclosure relates to compositions for treating hair and methods of using the compositions. The compositions comprise (a) citric acid and/or a salt thereof, and (b) glycine and/or a salt thereof. The compositions and methods may be for altering the color of the hair, and/or for providing strength and/or elasticity to the hair.

### CROSS-REFERENCE TO RELATED APPLICATIONS

This application claims priority to U.S. Provisional Application No. 63/548,778, filed Feb. 1, 2024, and FR Application No. 2404457, filed Apr. 29, 2024, both of which are incorporated herein in their entireties.

### TECHNICAL FIELD

The present disclosure relates to compositions for treating hair, for example for altering the color of hair, and methods of using the compositions for altering the color of the hair. The compositions and methods surprisingly maintain or enhance the fiber integrity and cosmetic properties of the treated hair.

### BACKGROUND

It is known that consumers desire to use cosmetic and care compositions that enhance the appearance of keratinous substrates such as hair, e.g., by changing the color, style, and/or shape of the hair, and/or by imparting various properties to hair, such as shine and conditioning. However, many of the known compositions and processes for enhancing the appearance of the hair involve harsh chemical treatment of the hair.

The process of altering the color of hair, for example, may involve lifting the color of the hair and/or depositing an artificial color onto the hair, which provides a different shade or color to the hair. The process of lifting the color of hair, also known as lightening, generally requires the use of compositions that comprise oxidizing agents. Such oxidizing agents enter the hair shaft and react with melanin in the hair, thereby lightening the color of the hair. Additional agents, such as persulfate salts, may be added to accelerate the process.

Imparting a color change or color effect on hair can be done using permanent, demi-permanent, semi-permanent, or temporary hair coloring products. Conventional permanent hair coloring products are dye compositions comprising oxidation dye precursors, which are also known as primary intermediates or couplers. These oxidation dye precursors are colorless or weakly colored compounds which, when combined with oxidizing or “developer” compositions, give rise to colored complexes by a process of oxidative condensation. The developer compositions conventionally use peroxides such as hydrogen peroxide as oxidizing agents.

Hair lightening or color lifting compositions and hair dyeing compositions possess an alkalinity such that these compositions have a pH value of above 7, typically 9 and above, and may generally require the presence of ammonia or an ammonia-generating compound, or other alkalizing agents such as monoethanolamine in amounts sufficient to make such compositions alkaline. These alkalizing agents cause the hair shaft to swell and open up the cuticle, thus allowing the small oxidative dye molecules to penetrate the cuticle and cortex before the oxidation condensation process is completed. The resulting larger-sized colored complexes from the oxidative reaction are then trapped inside the hair fiber, thereby permanently altering the color of the hair.

On the other hand, semi-permanent or temporary hair dyeing compositions typically use pigments, liposoluble dyes, or direct dyes which are deposited onto the hair fiber to impart color to the hair. Direct dyes are nonionic or ionic dyes and colored compounds capable of producing a more-or-less pronounced change of the natural color of the hair, resistant to shampoo-washing several times. These dyes may or may not be used with an oxidizing agent. In contrast with oxidation dye precursors, a direct dye is a relatively large molecule that does not penetrate easily into the core of the hair fiber.

While such compositions can effectively alter the color of hair, these chemical treatments are known to cause damage to the hair fibers and decrease the strength and elasticity of the hair, and negatively affect the sensorial properties, such as the smoothness, shine, and feel, of the hair. Indeed, consumers who have previously dyed hair often complain that their hair is difficult to comb, breaks easily, and generally feels rough and/or dull compared to before the dyeing process. Additionally, hair that is damaged during a bleaching and/or dyeing process may not take up color satisfactorily, resulting in unevenness or non-uniformity of color.

Thus, to reduce or avoid these drawbacks, new and additional ingredients and novel combinations of ingredients are needed that can help prevent, reduce, or even reverse damage when used before, during, after, or as a complement to compositions and processes for altering the color of the hair. Nevertheless, the choice of ingredients or combinations of ingredients could pose difficulties insofar as they cannot be detrimental to other cosmetic attributes such as ease and uniformity of application, rheology or viscosity properties of the compositions, stability of the compositions, and/or color deposit and target shade formation, and they cannot result in more disadvantages such as increased damage or a less healthy look or feel of the hair.

As such, there is a need for developing new and improved products that can be used for effectively altering the color of hair, while at the same time, maintaining or improving the integrity of hair during coloring the hair and avoiding or minimizing damage to the hair. The disclosure addresses these concerns and provides a surprisingly synergistic combination of components that, when used with hair color altering compositions, has been found to prevent, minimize, or eliminate damage to hair that would otherwise result from the hair dyeing process.

### SUMMARY

The disclosure relates to compositions and methods for treating keratin fibers, in particular hair. The compositions comprise components that surprisingly prevent or reduce damage to hair, provide strength and/or elasticity to hair, and/or permit the hair fibers to maintain integrity, for example in processes for altering the color of hair. In particular, the compositions comprise (a) at least one carboxylic acid and/or a salt thereof, (b) glycine and/or a salt thereof, (c) at least one solvent, and (d) optionally at least one hair color altering agent.

The disclosure relates to hair color base compositions and hair color altering compositions comprising (a) at least one carboxylic acid and/or a salt thereof, (b) glycine and/or a salt thereof, and (c) at least one solvent. In some embodiments, the weight ratio of the total amount of (b) glycine and salts thereof to the total amount of (a) carboxylic acids and salts thereof is greater than about 1, preferably ranging from about 1 to about 10, more preferably from about 1.25 to about 5, most preferably from about 1.5 to about 3.5. It has surprisingly and unexpectedly been found that the compositions provide integrity to keratin fibers and strengthen the fibers, and thus minimize and/or prevent damage to the hair such as that which may be caused by chemical treatments during processes for lifting or coloring the hair.

In various embodiments, the hair color base compositions comprise (a) at least one carboxylic acid and/or a salt thereof; (b) glycine and/or a salt thereof; (c) at least one solvent; and optionally, at least one additional component chosen from hair color altering agents, colloid agents, alkalizing agents, fatty alcohols, cationic polymers, or combinations of two or more thereof. The pH of the hair color base composition is typically greater than 7, such as from about 9 to about 11, from about 9.5 to about 11, or from about 10 to about 11. The total amount of carboxylic acids and salts thereof may range from about 0.01% to about 10%, preferably from about 0.05% to about 5%, more preferably from about 0.1% to about 3%, most preferably from about 0.1% to about 1% by weight, relative to the total weight of the composition. The total amount of glycine and salts thereof may range from about 0.01% to about 10%, preferably from about 0.05% to about 5%, more preferably from about 0.1% to about 3%, most preferably from about 0.2% to about 2% by weight, relative to the total weight of the composition. In some embodiments, the weight ratio of the total amount of (b) glycine and salts thereof to the total amount of (a) carboxylic acids and salts thereof is greater than about 1, such as greater than about 1.5, or greater than about 2, preferably ranging from about 1 to about 10, more preferably from about 1.25 to about 5, most preferably from about 1.5 to about 3.5. If present, the alkalizing agent may be chosen from organic and/or mineral alkalizing agents, for example monoethanolamine and/or ammonia or ammonium hydroxide. If present, the colloid agents may be chosen from, for example, celluloses and derivatives thereof, for example hydroxyethylcellulose and/or cetyl hydroxyethyl cellulose.

In further embodiments, the hair color base compositions comprise (a) at least one tricarboxylic acid and/or a salt thereof; (b) glycine and/or a salt thereof; (c) at least one solvent; and optionally, at least one additional component chosen from hair color altering agents, colloid agents, alkalizing agents, fatty alcohols, cationic polymers, or combinations of two or more thereof. The pH of the hair color base composition is typically greater than 7, such as from about 9 to about 11, from about 9.5 to about 11, or from about 10 to about 11. The total amount of tricarboxylic acids and salts thereof may range from about 0.01% to about 10%, preferably from about 0.05% to about 5%, more preferably from about 0.1% to about 3%, most preferably from about 0.1% to about 1% by weight, relative to the total weight of the composition. The total amount of glycine and salts thereof may range from about 0.01% to about 10%, preferably from about 0.05% to about 5%, more preferably from about 0.1% to about 3%, most preferably from about 0.2% to about 2% by weight, relative to the total weight of the composition. In some embodiments, the weight ratio of the total amount of (b) glycine and salts thereof to the total amount of (a) tricarboxylic acids and salts thereof is greater than about 1, such as greater than about 1.5, or greater than about 2, preferably ranging from about 1 to about 10, more preferably from about 1.25 to about 5, most preferably from about 1.5 to about 3.5. If present, the alkalizing agent may be chosen from organic and/or mineral alkalizing agents, for example monoethanolamine and/or ammonia or ammonium hydroxide. If present, the colloid agents may be chosen from, for example, celluloses and derivatives thereof, for example hydroxyethylcellulose and/or cetyl hydroxyethyl cellulose.

In still further embodiments, the hair color base compositions comprise (a) citric acid and/or a salt thereof; (b) glycine and/or a salt thereof; (c) at least one solvent; and optionally, at least one additional component chosen from hair color altering agents, colloid agents, alkalizing agents, fatty alcohols, cationic polymers, or combinations of two or more thereof. The pH of the hair color base composition is typically greater than 7, such as from about 9 to about 11, from about 9.5 to about 11, or from about 10 to about 11. The total amount of citric acid and salts thereof may range from about 0.01% to about 10%, preferably from about 0.05% to about 5%, more preferably from about 0.1% to about 3%, most preferably from about 0.1% to about 1% by weight, relative to the total weight of the composition. The total amount of glycine and salts thereof may range from about 0.01% to about 10%, preferably from about 0.05% to about 5%, more preferably from about 0.1% to about 3%, most preferably from about 0.2% to about 2% by weight, relative to the total weight of the composition. In some embodiments, the weight ratio of the total amount of (b) glycine and salts thereof to the total amount of (a) citric acid and salts thereof is greater than about 1, such as greater than about 1.5, or greater than about 2, preferably ranging from about 1 to about 10, more preferably from about 1.25 to about 5, most preferably from about 1.5 to about 3.5. If present, the alkalizing agent may be chosen from organic and/or mineral alkalizing agents, for example monoethanolamine and/or ammonia or ammonium hydroxide. If present, the colloid agents may be chosen from, for example, celluloses and derivatives thereof, for example hydroxyethylcellulose and/or cetyl hydroxyethyl cellulose.

In a particularly preferred embodiment, the hair color base composition comprises (a) from about 0.03% to about 1%, such as from about 0.05% to about 0.5%, of citric acid and/or a salt thereof, (b) from about 0.1% to about 1.5%, such as from about 0.2% to about 1.1%, of glycine and/or a salt thereof, (c) a solvent comprising water and optionally one or more non-aqueous solvents, (d) one or more oxidative dye compounds, (e) one or more colloid agents, preferably including cellulose and/or a derivative thereof, more preferably including hydroxyethylcellulose and/or cetyl hydroxyethyl cellulose, and (f) from about 0.5% to about 10%, such as from about 1% to about 8% or from about 2% to about 6%, of one or more alkalizing agents, preferably including monoethanolamine. The weight ratio of the total amount of (b) glycine and salts thereof to the total amount of (a) citric acid and salts thereof in the hair color base composition is preferably greater than about 1, such as greater than about 1.5, or greater than about 2, preferably ranging from about 1 to about 10, more preferably from about 1.25 to about 5, most preferably from about 1.5 to about 3.5. In preferred embodiments, hair color base composition comprises at least one additional component chosen from fatty alcohols, surfactants, cationic polymers, fatty compounds other than fatty alcohols, or combinations of two or more thereof, particularly fatty alcohols.

The disclosure also relates to hair color altering compositions comprising a hair color base composition as described herein and at least one oxidizing agent. For example, the hair color altering composition may be prepared by mixing a hair color base composition according to the disclosure with a developer composition comprising one or more oxidizing agents, at or near the time of use. In various embodiments, the oxidizing composition is a 5V, 10V, 20V, 30V, or 40V hydrogen peroxide developer composition. In various embodiments, the hair color base composition and the oxidizing composition may be mixed at a mixing ratio ranging from 5:1 to 1:5, preferably ranging from 3:1 to 1:3, more preferably ranging from 2:1 to 1:2, most preferably ranging from 1.5:1 to 1:1.5. The hair color altering compositions may, in various embodiments, be permanent or demi-permanent.

In further embodiments, the disclosure relates to methods of making a hair color altering composition, the methods comprising mixing a hair color base composition according to the disclosure with a developer composition comprising one or more oxidizing agents. In various embodiments, the oxidizing composition is a 5V, 10V, 20V, 30V, or 40V hydrogen peroxide developer composition. For example, the hair color base composition and the oxidizing composition may be mixed at a mixing ratio ranging from 5:1 to 1:5, preferably ranging from 3:1 to 1:3, more preferably ranging from 2:1 to 1:2, most preferably ranging from 1.5:1 to 1:1.5.

In still further embodiments, the disclosure relates to methods of treating keratin fibers, for example hair, by applying a hair color altering composition as described here, e.g. prepared by mixing a hair color base composition according to the disclosure with a developer composition comprising one or more oxidizing agents, at or near the time of use, to the hair. The hair color altering composition may be left to process on the hair for a desired period of time, for example from about 1 minute to about 60 minutes, from about 5 minutes to about 45 minutes, or from about 10 minutes to about 30 minutes. The hair may subsequently be rinsed.

In further embodiments still, the disclosure relates to kits comprising a hair color base composition as described herein. For example, a kit may comprise a first compartment or container comprising a hair color base composition and a second compartment or container, which may, for example, comprise a developer composition.

### DETAILED DESCRIPTION

The present disclosure relates to compositions for altering the color of hair, including both hair color base compositions and hair color altering compositions comprising a hair color base composition according to the disclosure and at least one oxidizing agent, as well as kits containing the compositions and methods of using the compositions. A unique feature of the compositions and methods according to the present disclosure is a bonding system incorporated in the compositions for altering the color of the hair, where the bonding system comprises a synergistic combination of one or more alpha-hydroxy acids comprising at least three hydrogen donor carboxyl groups and/or salts thereof, and at least one amino acid chosen from glycine and/or salts thereof. It has been surprisingly found that the synergistic combination of bonding agents can significantly enhance hair fiber integrity and/or minimize hair damage caused by oxidative hair dye compositions and processes.

### I. Compositions

In various embodiments, the compositions according to the disclosure are compositions for altering the color of hair, for example for demi-permanently or permanently altering the hair color. The compositions may be hair color altering base compositions that can be mixed with a developer composition comprising at least one oxidizing agent at or near the time of use, or may be hair color altering compositions that result from the mixture of a hair color altering base composition and a developer composition comprising at least one oxidizing agent.

### Hair Color Base Compositions

In various embodiments, hair color base compositions (referred to interchangeably as “hair base compositions” or “base compositions”) according to the disclosure comprise a synergistic combination of (a) at least one carboxylic acid and/or a salt thereof, (b) glycine and/or a salt thereof, and (c) at least one solvent. In various embodiments, the hair color base compositions further comprise at least one additional component chosen from oxidative dye compounds, direct dyes, pigments, fatty alcohols, fatty compounds other than fatty alcohols, surfactants, cationic polymers, alkalizing agents, colloid agents, auxiliary agents, or combinations of two or more thereof. The pH of the hair color base composition is typically greater than 7.

### Carboxylic Acids

Hair color base compositions according to the disclosure include at least one carboxylic acid. According to various embodiments of the disclosure, useful carboxylic acids include organic compounds that have, for example, one (mono-), two (di-), three (tri-), or more acid functional groups and at least one carbon atom. The carboxylic acids are preferably non-polymeric. In preferred embodiments, compositions according to the disclosure include at least one tricarboxylic acid, in particular citric acid and/or a salt thereof.

Carboxylic acids that can be used may optionally have a molecular weight of less than about 500 g/mol, less than about 400 g/mol, less than about 300 g/mol, or less than about 200 g/mol. In preferred embodiments, the carboxylic acids have a molecular weight of less than about 300 g/mol, or less than about 200 g/mol.

Salts of carboxylic acids can also be used, and are expressly included when the term “carboxylic acid” is used unless expressly stated otherwise. The salts include salts with organic or mineral bases, for example the salts of alkali metals, such as the lithium, sodium, or potassium salts; the salts of alkaline earth metals, such as the magnesium or calcium salts, and the zinc salts. The alkali metal or alkaline earth metal salts are preferred in some embodiments, and in particular the sodium salts.

In some embodiments, the compositions comprise one or more hydroxylated (poly)carboxylic acids comprising from 2 to 12 carbon atoms, such as from 3 to 10 carbon atoms, or from 4 to 8 carbon atoms, and may be saturated or unsaturated, and linear or branched, and/or a salt thereof. Useful (poly)acids comprise at least one-COOH group (in acid or salified form); they can thus comprise a single —COOH group (monoacid) or can comprise more than one, for example two —COOH groups (in acid or salified form), or two or three —COOH groups (in acid or salified form) (polyacids). Useful (poly)acids also comprise at least one —OH group, such as from one to three or from two to three —OH groups, for example one, two, or three —OH groups. Optionally, the (poly)acids comprise in total from 2 to 10 or from 3 to 8 carbon atoms, and from two to three —OH groups, and their hydrocarbon chain is saturated or unsaturated, linear or branched, and preferably saturated and linear. In some embodiments, the hydroxylated (poly)carboxylic acids and/or their salts comprise in total from 4 to 8 carbon atoms, from one to three —OH groups, and from two to three-COOH groups (in acid or salified form). Unless stated otherwise, as used herein the term “(poly)acid” includes both monoacids and polyacids.

Non-limiting examples of monocarboxylic acids that can be chosen include formic acid, acetic acid, propionic acid, butyric acid, valeric acid, caproic acid, enanthic acid, caprylic acid, lactic acid, salts thereof, or combinations of two or more thereof.

Non-limiting examples of dicarboxylic acids that can be chosen include oxalic acid, malonic acid, malic acid, glutaric acid, citraconic acid, succinic acid, adipic acid, tartaric acid, fumaric acid, maleic acid, salts thereof, or combinations of two or more thereof.

Non-limiting examples of tricarboxylic acids include citric acid, isocitric acid, aconitic acid, propane-1,2,3-tricarboxylic acid, salts thereof, or combinations of two or more thereof. In some embodiments, the carboxylic acid may comprise, consist essentially of, or consist of tricarboxylic acids and/or salts thereof, in particular citric acid and/or a salt thereof.

In some embodiments, the carboxylic acid(s) may be chosen from oxalic acid, malonic acid, glutaric acid, succinic acid, adipic acid, glycolic acid, citric acid, tartaric acid, malic acid, maleic acid, lactic acid, salts thereof, or combinations of two or more thereof. In particular embodiments, the carboxylic acids are chosen from α-hydroxy acids and their salts, and in particular from lactic acid, glycolic acid, tartaric acid, malic acid, citric acid, or salts thereof, in particular alkali metal or alkaline earth metal salts. It may in some embodiments be particularly advantageous to choose citric acid, lactic acid, malic acid, and/or tartaric acid and/or their salts, in particular alkali metal or alkaline earth metal salts, such as sodium citrate and/or sodium tartrate; preferably citric acid and/or its salts, in particular alkali metal or alkaline earth metal salts, such as sodium citrate.

The total amount of carboxylic acids and salts thereof may range from about 0.01% to about 10%, such as about 0.025% to about 5%, about 0.05% to about 3%, about 0.05% to about 2%, about 0.075% to about 1%, about 0.1% to about 0.5%, or about 0.15% to about 0.25% by weight, relative to the total weight of the hair color base composition. For example, in various embodiments, the total amount of carboxylic acids and salts thereof may range from about 0.01% to about 5%, such as about 0.01% to about 4.5%, about 0.01% to about 4%, about 0.01% to about 3.5%, about 0.01% to about 3%, about 0.01% to about 2.5%, about 0.01% to about 2%, about 0.01% to about 1.5%, about 0.01% to about 1.4%, about 0.01% to about 1.3%, about 0.01% to about 1.2%, about 0.01% to about 1.1%, about 0.01% to about 1%, about 0.01% to about 0.9%, about 0.01% to about 0.8%, about 0.01% to about 0.7%, about 0.01% to about 0.6%, about 0.01% to about 0.5%, about 0.01% to about 0.4%, about 0.01% to about 0.3%, about 0.01% to about 0.2%, about 0.01% to about 0.1%, about 0.05% to about 5%, about 0.05% to about 4.5%, about 0.05% to about 4%, about 0.05% to about 3.5%, about 0.05% to about 3%, about 0.05% to about 2.5%, about 0.05% to about 2%, about 0.05% to about 1.5%, about 0.05% to about 1.4%, about 0.05% to about 1.3%, about 0.05% to about 1.2%, about 0.05% to about 1.1%, about 0.05% to about 1%, about 0.05% to about 0.9%, about 0.05% to about 0.8%, about 0.05% to about 0.7%, about 0.05% to about 0.6%, about 0.05% to about 0.5%, about 0.05% to about 0.4%, about 0.05% to about 0.3%, about 0.05% to about 0.2%, about 0.05% to about 0.1%, about 0.1% to about 5%, about 0.1% to about 4.5%, about 0.1% to about 4%, about 0.1% to about 3.5%, about 0.1% to about 3%, about 0.1% to about 2.5%, about 0.1% to about 2%, about 0.1% to about 1.5%, about 0.1% to about 1.4%, about 0.1% to about 1.3%, about 0.1% to about 1.2%, about 0.1% to about 1.1%, about 0.1% to about 1%, about 0.1% to about 0.9%, about 0.1% to about 0.8%, about 0.1% to about 0.7%, about 0.1% to about 0.6%, about 0.1% to about 0.5%, about 0.1% to about 0.4%, about 0.1% to about 0.3%, about 0.1% to about 0.2%, about 0.15% to about 5%, about 0.15% to about 4.5%, about 0.15% to about 4%, about 0.15% to about 3.5%, about 0.15% to about 3%, about 0.15% to about 2.5%, about 0.15% to about 2%, about 0.15% to about 1.5%, about 0.15% to about 1.4%, about 0.15% to about 1.3%, about 0.15% to about 1.2%, about 0.15% to about 1.1%, about 0.15% to about 1%, about 0.15% to about 0.9%, about 0.15% to about 0.8%, about 0.15% to about 0.7%, about 0.15% to about 0.6%, about 0.15% to about 0.5%, about 0.15% to about 0.4%, about 0.15% to about 0.3%, or about 0.15% to about 0.2% by weight, relative to the total weight of the hair color base composition. In various embodiments, the total amount of carboxylic acids and salts thereof is about or exactly 0.05%, about or exactly 0.075%, about or exactly 0.1%, about or exactly 0.15%, about or exactly 0.2%, about or exactly 0.25%, about or exactly 0.3%, about or exactly 0.35%, or about or exactly 0.4% by weight, relative to the total weight of the hair color base composition, including all ranges and subranges thereof.

In some embodiments, the hair color base composition may comprise one or more tricarboxylic acids and/or salts thereof, preferably citric acid and/or one or more salts thereof, in an amount ranging from about 0.01% to about 10%, such as about 0.025% to about 5%, about 0.05% to about 3%, about 0.05% to about 2%, about 0.075% to about 1%, about 0.1% to about 0.5%, or about 0.15% to about 0.25% by weight, relative to the total weight of the hair color base composition. In various embodiments, the total amount of citric acid and salts thereof is about or exactly 0.05%, about or exactly 0.075%, about or exactly 0.1%, about or exactly 0.15%, about or exactly 0.2%, about or exactly 0.25%, about or exactly 0.3%, about or exactly 0.35%, or about or exactly 0.4% by weight, relative to the total weight of the hair color base composition, including all ranges and subranges thereof.

### Glycine

It has been discovered that glycine, in combination with one or more carboxylic acids described above, in particular with one or more tricarboxylic acids and salts thereof, provides unexpectedly superior benefits for maintaining or enhancing the integrity and cosmetic properties of hair when used in or with compositions and methods for altering the color of hair. Thus, compositions according to the disclosure include glycine and/or one or more salts thereof. Salts of glycine are expressly included when the term “glycine” is used, unless expressly stated otherwise.

In various embodiments, the total amount of glycine and salts thereof ranges from about 0.01% to about 10%, such as about 0.025% to about 5%, about 0.05% to about 3%, about 0.075% to about 2%, about 0.1% to about 1%, or about 0.25% to about 0.75% by weight, relative to the total weight of the hair color base composition. For example, in various embodiments, the total amount of carboxylic acids and salts thereof may range from about 0.1% to about 5%, such as from about 0.1% to about 4.5%, about 0.1% to about 4%, about 0.1% to about 3.5%, about 0.1% to about 3%, about 0.1% to about 2.5%, about 0.1% to about 2%, about 0.1% to about 1.5%, about 0.1% to about 1.4%, about 0.1% to about 1.3%, about 0.1% to about 1.2%, about 0.1% to about 1.1%, about 0.1% to about 1%, about 0.1% to about 0.9%, about 0.1% to about 0.8%, about 0.1% to about 0.7%, about 0.1% to about 0.6%, about 0.1% to about 0.5%, about 0.1% to about 0.4%, about 0.1% to about 0.3%, about 0.2% to about 5%, about 0.2% to about 4.5%, about 0.2% to about 4%, about 0.2% to about 3.5%, about 0.2% to about 3%, about 0.2% to about 2.5%, about 0.2% to about 2%, about 0.2% to about 1.5%, about 0.2% to about 1.4%, about 0.2% to about 1.3%, about 0.2% to about 1.2%, about 0.2% to about 1.1%, about 0.2% to about 1%, about 0.2% to about 0.9%, about 0.2% to about 0.8%, about 0.2% to about 0.7%, about 0.2% to about 0.6%, about 0.2% to about 0.5%, about 0.2% to about 0.4%, about 0.2% to about 0.3%, about 0.3% to about 5%, about 0.3% to about 4.5%, about 0.3% to about 4%, about 0.3% to about 3.5%, about 0.3% to about 3%, about 0.3% to about 2.5%, about 0.3% to about 2%, about 0.3% to about 1.5%, about 0.3% to about 1.4%, about 0.3% to about 1.3%, about 0.3% to about 1.2%, about 0.3% to about 1.1%, about 0.3% to about 1%, about 0.3% to about 0.9%, about 0.3% to about 0.8%, about 0.3% to about 0.7%, about 0.3% to about 0.6%, about 0.3% to about 0.5%, about 0.3% to about 0.4%, about 0.4% to about 5%, about 0.4% to about 4.5%, about 0.4% to about 4%, about 0.4% to about 3.5%, about 0.4% to about 3%, about 0.4% to about 2.5%, about 0.4% to about 2%, about 0.4% to about 1.5%, about 0.4% to about 1.4%, about 0.4% to about 1.3%, about 0.4% to about 1.2%, about 0.4% to about 1.1%, about 0.4% to about 1%, about 0.4% to about 0.9%, about 0.4% to about 0.8%, about 0.4% to about 0.7%, about 0.4% to about 0.6%, about 0.4% to about 0.5%, about 0.5% to about 5%, about 0.5% to about 4.5%, about 0.5% to about 4%, about 0.5% to about 3.5%, about 0.5% to about 3%, about 0.5% to about 2.5%, about 0.5% to about 2%, about 0.5% to about 1.5%, about 0.5% to about 1.4%, about 0.5% to about 1.3%, about 0.5% to about 1.2%, about 0.5% to about 1.1%, about 0.5% to about 1%, about 0.5% to about 0.9%, about 0.5% to about 0.8%, about 0.5% to about 0.7%, or about 0.5% to about 0.6% by weight, relative to the total weight of the hair color base composition. In various embodiments, the total amount of glycine and salts thereof is about or exactly 0.1%, about or exactly 0.2%, about or exactly 0.3%, about or exactly 0.35%, about or exactly 0.4%, about or exactly 0.45%, about or exactly 0.5%, about or exactly 0.55%, about or exactly 0.6%, about or exactly 0.65%, about or exactly 0.7%, about or exactly 0.75%, or about or exactly 0.8% by weight, relative to the total weight of the hair color base composition, including all ranges and subranges thereof.

It has been discovered that by choosing amounts of carboxylic acids and glycine relative to each other, the hair fiber integrity is surprisingly improved. Therefore, in various embodiments, the amounts of carboxylic acids and glycine may be chosen such that the weight ratio of the total amount of glycine and salts thereof to the total amount of carboxylic acids and salts thereof is greater than or equal to about 1, such as greater than or equal to about 1.25, greater than or equal to about 1.5, greater than or equal to about 1.75, greater than or equal to about 2, greater than or equal to about 2.25, or greater than or equal to about 2.5. For instance, in certain embodiments, the weight ratio of the total amount of glycine and salts thereof to the total amount of carboxylic acids and salts thereof ranges from about 1 to about 10, such as from about 1 to about 7.5, from about 1 to about 5, from about 1 to about 4, from about 1 to about 3, from about 1 to about 2.75, from about 1 to about 2.5, from about 1 to about 2.25, from about 1 to about 2, from about 1.25 to about 10, from about 1.25 to about 7.5, from about 1.25 to about 5, from about 1.25 to about 4, from about 1.25 to about 3, from about 1.25 to about 2.75, from about 1.25 to about 2.5, from about 1.25 to about 2.25, from about 1.25 to about 2, from about 1.5 to about 10, from about 1.5 to about 7.5, from about 1.5 to about 5, from about 1.5 to about 4, from about 1.5 to about 3, from about 1.5 to about 2.75, from about 1.5 to about 2.5, from about 1.5 to about 2.25, from about 1.5 to about 2, from about 1.75 to about 10, from about 1.75 to about 7.5, from about 1.75 to about 5, from about 1.75 to about 4, from about 1.75 to about 3, from about 1.75 to about 2.75, from about 1.75 to about 2.5, from about 1.75 to about 2.25, from about 1.75 to about 2, from about 2 to about 10, from about 2 to about 7.5, from about 2 to about 5, from about 2 to about 4, from about 2 to about 3, from about 2 to about 2.75, from about 2 to about 2.5, from about 2 to about 2.25, from about 2.25 to about 10, from about 2.25 to about 7.5, from about 2.25 to about 5, from about 2.25 to about 4, from about 2.25 to about 3, from about 2.25 to about 2.75, or from about 2.25 to about 2.5. In some embodiments, the weight ratio of the total amount of glycine and salts thereof to the total amount of carboxylic acids and salts thereof is about or exactly 1, about or exactly 1.1, about or exactly 1.2, about or exactly 1.3, about or exactly 1.4, about or exactly 1.5, about or exactly 1.6, about or exactly 1.7, about or exactly 1.8, about or exactly 1.9, about or exactly 2, about or exactly 2.1, about or exactly 2.2, about or exactly 2.3, about or exactly 2.4, about or exactly 2.5, about or exactly 2.6, about or exactly 2.7, about or exactly 2.8, about or exactly 2.9, about or exactly 3, about or exactly 3.1, about or exactly 3.2, about or exactly 3.3, about or exactly 3.4, or about or exactly 3.5, including all ranges and subranges thereof.

By way of non-limiting example, hair color base compositions according to the disclosure may comprise glycine and one or more tricarboxylic acids, e.g. citric acid, wherein the total amounts of the amounts of tricarboxylic acids and salts thereof, e.g. citric acid and salts thereof, and glycine and salts thereof, are chosen such that the weight ratio of the total amount of glycine and salts thereof to the total amount of tricarboxylic acids and salts thereof, e.g. citric acid and salts thereof, is greater than or equal to about 1, such as greater than or equal to about 1.25, greater than or equal to about 1.5, greater than or equal to about 1.75, greater than or equal to about 2, greater than or equal to about 2.25, or greater than or equal to about 2.5, for example ranges from about 1 to about 10, from about 1.25 to about 7.5, from about 1.5 to about 5, from about 1.75 to about 4, from about 2 to about 3, or from about 2.25 to about 2.75.

### Hair Color Altering Agents

Compositions according to the disclosure may be useful for permanently or demi-permanently altering the color of the hair. Thus, hair color base compositions may further comprise at least one agent for altering the color of the hair, in an amount sufficient to alter the color of the hair. Hair color altering agents can be chosen from agents that permanently or non-permanently alter the color of the hair. Non-limiting examples of permanent hair color altering agents include oxidative dye compounds and oxidizing agents, and non-limiting examples of non-permanent hair color altering agents include direct dyes. Preferably, the compositions comprise at least one agent for permanently or demi-permanently altering the color of the hair. However, it should be noted that compositions according to the disclosure may be free or substantially free of hair color altering agents, but may still provide benefits such as shine to the hair, in addition to hair fiber strength and/or elasticity, and/or other cosmetic benefits as described herein. Thus, unless a composition expressly includes a hair color altering agent, in an amount sufficient to alter the color of the hair, the composition should not be construed as requiring a hair color altering effect even if referred to as a “hair color base” or “hair color altering” composition.

Oxidative dye compounds include dye precursors (also referred to as oxidation bases) and couplers. The oxidation dye compounds may be used in the form of salts, solvates, and/or solvates of salts.

The addition salts of the oxidation dye compounds present in the composition according to the invention can be chosen from the addition salts with an acid, such as the hydrochlorides, hydrobromides, sulfates, citrates, succinates, tartrates, lactates, tosylates, benzenesulfonates, methanesulfonates, phosphates and acetates, and the addition salts with a base such as sodium hydroxide, potassium hydroxide, aqueous ammonia, amines, or alkanolamines.

Moreover, the solvates of the oxidation dye compounds more particularly represent the hydrates of said oxidation bases and/or the combination of said oxidation bases with a linear or branched C1-C4 alcohol such as methanol, ethanol, isopropanol, or n-propanol. In some embodiments, the solvates are hydrates.

The compositions comprise one or more oxidation bases when the composition is a composition for permanently or demi-permanently dyeing or coloring the hair. Useful and non-limiting oxidation bases include para-phenylenediamines, bis(phenyl)alkylenediamines, para-aminophenols, ortho-aminophenols, and heterocyclic bases, and the addition salts thereof, the solvates thereof, and solvates of the salts thereof.

Among the para-phenylenediamines, non-limiting examples that may be mentioned include para-phenylenediamine, para-toluenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-N,N-bis(β-hydroxyethyl)amino-2-methylaniline, 4-N, N-bis(3-hydroxyethyl)amino-2-chloroaniline, 2-β-hydroxyethyl-para-phenylenediamine, 2-methoxymethyl-para-phenylenediamine, 2-γ-hydroxypropyl-para-phenylenediamine; 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N-ethyl-N-(β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylaminoethyloxy-para-phenylenediamine, N-(β-methoxyethyl)-para-phenylenediamine, 4-aminophenylpyrrolidine, 2-thienyl-para-phenylenediamine, 2-β-hydroxyethylamino-5-aminotoluene and 3-hydroxy-1-(4′-aminophenyl) pyrrolidine, and the addition salts thereof, the solvates thereof, and solvates of the salts thereof.

Among the para-phenylenediamines mentioned above, para-phenylenediamine, para-toluenediamine, 2-isopropyl-para-phenylenediamine, 2-β-hydroxyethyl-para-phenylenediamine, 2-methoxymethyl-para-phenylenediamine, 2-γ-hydroxypropyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine and 2-β-acetylaminoethyloxy-para-phenylenediamine, and the addition salts thereof, the solvates thereof, and solvates of the salts thereof are particularly useful.

Among the bis(phenyl)alkylenediamines, non-limiting examples that may be mentioned include N,N′-bis(β-hydroxyethyl)-N,N′-bis(4′-aminophenyl)-1,3-diaminopropanol, N,N′-bis(β-hydroxyethyl)-N, N′-bis(4′-aminophenyl)ethylenediamine, N,N′-bis(4-aminophenyl)tetramethylenediamine, N,N′-bis(β-hydroxyethyl)-N, N′-bis(4-aminophenyl)tetramethylenediamine, N,N′-bis(4-methylaminophenyl)tetramethylenediamine, N,N′-bis(ethyl)-N, N′-bis(4′-amino-3′-methylphenyl)ethylenediamine and 1,8-bis(2,5-diaminophenoxy)-3,6-dioxaoctane, and the addition salts thereof, the solvates thereof, and solvates of the salts thereof.

Among the para-aminophenols, non-limiting examples that may be mentioned include para-aminophenol, 4-amino-3-methylphenol, 4-amino-3-fluorophenol, 4-amino-3-chlorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl) phenol and 4-amino-2-fluorophenol, and the addition salts thereof, the solvates thereof, and solvates of the salts thereof.

Among the ortho-aminophenols, non-limiting examples that may be mentioned include 2-aminophenol, 2-amino-5-methylphenol, 2-amino-6-methylphenol and 5-acetamido-2-aminophenol, and the addition salts thereof, solvates thereof and solvates of the salts thereof.

Among the heterocyclic bases, non-limiting examples that may be mentioned include pyridine derivatives, pyrimidine derivatives and pyrazole derivatives.

Among the pyridine derivatives, non-limiting mention may be made of 2,5-diaminopyridine, 2-(4-methoxyphenyl)amino-3-aminopyridine, 3,4-diaminopyridine, and the addition salts thereof, the solvates thereof, and solvates of the salts thereof.

Other pyridine oxidation bases that are useful in the present invention are the 3-aminopyrazolo[1,5-a]pyridine oxidation bases or their addition salts described. Non-limiting examples that may be mentioned include pyrazolo[1,5-a]pyrid-3-ylamine, 2-acetylaminopyrazolo[1,5-a]pyrid-3-ylamine, 2-(morpholin-4-yl)pyrazolo[1,5-a]pyrid-3-ylamine, 3-aminopyrazolo[1,5-a]pyridine-2-carboxylic acid, 2-methoxypyrazolo[1,5-a]pyrid-3-ylamine, (3-aminopyrazolo[1,5-a]pyrid-7-yl)methanol, 2-(3-aminopyrazolo[1,5-a]pyrid-5-yl)ethanol, 2-(3-aminopyrazolo[1,5-a]pyrid-7-yl)ethanol, (3-aminopyrazolo[1,5-a]pyrid-2-yl)methanol, 3,6-diaminopyrazolo[1,5-a]pyridine, 3,4-diaminopyrazolo[1,5-a]pyridine, pyrazolo[1,5-a]pyridine-3,7-diamine, 7-(morpholin-4-yl)pyrazolo[1,5-a]pyrid-3-ylamine, pyrazolo[1,5-a]pyridine-3,5-diamine, 5-(morpholin-4-yl)pyrazolo[1,5-a]pyrid-3-ylamine, 2-[(3-aminopyrazolo[1,5-a]pyrid-5-yl) (2-hydroxyethyl)amino]ethanol, 2-[(3-aminopyrazolo[1,5-a]pyrid-7-yl) (2-hydroxyethyl)amino]ethanol, 3-aminopyrazolo[1,5-a]pyridin-5-ol, 3-aminopyrazolo[1,5-a]pyridin-4-ol, 3-aminopyrazolo[1,5-a]pyridin-6-ol, 3-aminopyrazolo[1,5-a]pyridin-7-ol and 2-(3-amino-pyrazolo[1,5-a]pyridin-2-yl)oxoethanol, and the addition salts thereof, the solvates thereof, and solvates of the salts thereof.

Among the pyrimidine derivatives, non-limiting mention may be made of 2,4,5,6-tetraaminopyrimidine, 4-hydroxy-2,5,6-triaminopyrimidine, 2-hydroxy-4,5,6-triaminopyrimidine, 2,4-dihydroxy-5,6-diaminopyrimidine, 2,5,6-triaminopyrimidine and the addition salts thereof, and the tautomeric forms thereof when a tautomeric equilibrium exists.

Non-limiting examples of pyrazole derivatives that may be mentioned include 4,5-diamino-1-methylpyrazole, 4,5-diamino-1-(β-hydroxyethyl) pyrazole, 3,4-diaminopyrazole, 4,5-diamino-1-(4′-chlorobenzyl) pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl) pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole and 3,5-diamino-4-(β-hydroxyethyl)amino-1-methylpyrazole, and the addition salts thereof, the solvates thereof, and solvates of the salts thereof. Use may also be made of 4,5-diamino-1-(β-methoxyethyl) pyrazole.

Non-limiting pyrazole derivatives that may be mentioned include diamino-N,N-dihydropyrazolopyrazolones and the following compounds and the addition salts thereof: 2,3-diamino-6,7-dihydro-1H,5H-pyrazolo[1,2-a]pyrazol-1-one, 2-amino-3-ethylamino-6,7-dihydro-1H,5H-pyrazolo[1,2-a]pyrazol-1-one, 2-amino-3-isopropylamino-6,7-dihydro-1H,5H-pyrazolo[1,2-a]pyrazol-1-one, 2-amino-3-(pyrrolidin-1-yl)-6,7-dihydro-1H,5H-pyrazolo[1,2-a]pyrazol-1-one, 4,5-diamino-1,2-dimethyl-1,2-dihydropyrazol-3-one, 4,5-diamino-1,2-diethyl-1,2-dihydropyrazol-3-one, 4,5-diamino-1,2-bis(2-hydroxyethyl)-1,2-dihydropyrazol-3-one, 2-amino-3-(2-hydroxyethyl)amino-6,7-dihydro-1H,5H-pyrazolo[1,2-a]pyrazol-1-one, 2-amino-3-dimethylamino-6,7-dihydro-1H,5H-pyrazolo[1,2-a]pyrazol-1-one, 2,3-diamino-5,6,7,8-tetrahydro-1H,6H-pyridazino[1,2-a]pyrazol-1-one, 4-amino-1,2-diethyl-5-(pyrrolidin-1-yl)-1,2-dihydropyrazol-3-one, 4-amino-5-(3-dimethylaminopyrrolidin-1-yl)-1,2-diethyl-1,2-dihydropyrazol-3-one, 2,3-diamino-6-hydroxy-6,7-dihydro-1H,5H-pyrazolo[1,2-a]pyrazol-1-one, the salts thereof, the solvates thereof, and solvates of the salts thereof.

Heterocyclic bases that can be used include, for example, 4,5-diamino-1-(β-hydroxyethyl) pyrazole and/or 2,3-diamino-6,7-dihydro-1H,5H-pyrazolo[1,2-a]pyrazol-1-one and/or 2-(3-aminopyrazolo[1,5-a]pyridin-2-yl)oxoethanol and/or a salt thereof, a solvate thereof, and a solvate of the salts thereof.

In some particular embodiments, the oxidation base(s) are chosen from para-phenylenediamines, bis(phenyl)alkylenediamines, para-aminophenols, ortho-aminophenols, heterocyclic bases, and the corresponding addition salts, the solvates thereof and/or the solvates of the salts thereof, and mixtures thereof; more preferably from 2-methoxymethyl-para-phenylenediamine, 2-β-hydroxyethyl-para-phenylenediamine, 2-γ-hydroxypropyl-para-phenylenediamine, and the addition salts thereof, the solvates thereof and/or the solvates of the salts thereof, and mixtures thereof.

In another embodiment, the hair color base composition is free of oxidation bases chosen from para-phenylenediamine, para-toluenediamine, addition salts thereof, solvates thereof and solvates of the salts thereof.

Typically, the oxidation bases are present in a hair color base composition for permanently or demi-permanently altering the color of hair in a total amount ranging from about 0.0001% to about 10%, from about 0.005% to about 7%, or from about 0.1% to about 4% by weight, relative to the total weight of the hair color base composition.

The hair color base composition may also comprise one or more couplers, which may be chosen from the couplers conventionally used for the dyeing of keratin fibers. In at least some embodiments, hair color base compositions for permanently or demi-permanently altering the color of hair comprise one or more couplers in addition to one or more oxidation bases.

In some embodiments, the couplers are chosen from meta-phenylenediamines, meta-aminophenols, meta-diphenols, naphthalene-based couplers and heterocyclic couplers, and also the addition salts thereof, and/or the solvates thereof, and/or solvates of the salts thereof.

Non-limiting examples that may be mentioned include 1,3-dihydroxybenzene, 1,3-dihydroxy-2-methylbenzene, 4-chloro-1,3-dihydroxybenzene, 1-hydroxy-3-aminobenzene, 1-methyl-2-hydroxy-4-β-hydroxyethylaminobenzene, 4-amino-2-hydroxytoluene, 5-amino-6-chloro-2-methylphenol, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, 3-ureidoaniline, 3-ureido-1-dimethylaminobenzene, sesamol, 1-β-hydroxyethylamino-3,4-methylenedioxybenzene, α-naphthol, 2-methyl-1-naphthol, 6-hydroxyindole, 4-hydroxyindole, 4-hydroxy-N-methylindole, 5-methoxy-6-hydroxyindole, 2-amino-3-hydroxypyridine, 6-hydroxybenzomorpholine, 2-amino-4-hydroxyethylaminoanisole, 3-amino-6-methoxy-2-methylaminopyridine, 3,5-diamino-2,6-dimethoxypyridine, 1-N-(β-hydroxyethyl)amino-3,4-methylenedioxybenzene, 2,6-bis(β-hydroxyethylamino)toluene, 6-hydroxyindoline, 2,6-dihydroxy-4-methylpyridine, 2-chloro-3,5-diaminopyridine, 2-chloro-3,5-diamino-6-methoxypyridine, 2-chloro-3,5-diamino-6-methylpyridine, 1-H-3-methylpyrazol-5-one, 1-phenyl-3-methylpyrazol-5-one, 4-(3,5-diaminopyridin-2-yl)-1-(2-hydroxyethyl)-1-methylpiperazin-1-ium chloride, 2,6-dimethylpyrazolo[1,5-b]-1,2,4-triazole, 2,4,6-trimethoxyaniline hydrochloride, 2,6-dimethyl[3,2-c]-1,2,4-triazole, 6-methylpyrazolo[1,5-a]benzimidazole and 2,6-diaminopyrazine, the addition salts thereof, and/or the solvates thereof, and/or solvates of the salts thereof, and mixtures thereof.

In some examples, the coupler(s) are chosen from 1,3-dihydroxybenzene, 1,3-dihydroxy-2-methylbenzene, 4-chloro-1,3-dihydroxybenzene, 1-hydroxy-3-aminobenzene, 1-methyl-2-hydroxy-4-β-hydroxyethylaminobenzene, 4-amino-2-hydroxytoluene, 5-amino-6-chloro-2-methylphenol, 2,4-diamino-1-(β-hydroxyethyloxy)benzene, α-naphthol, 6-hydroxyindole, 2-amino-3-hydroxypyridine, 6-hydroxybenzomorpholine, 3-amino-6-methoxy-2-methylaminopyridine, 2-amino-4-hydroxyethylaminoanisole, hydroxyethyl-3,4-methylenedioxyaniline and 2-amino-5-ethylphenol, the addition salts thereof, and/or the solvates thereof, and/or solvates of the salts thereof, and mixtures thereof.

For example, in some embodiments couplers are chosen from 3-amino-6-methoxy-2-methylaminopyridine, 6-hydroxybenzomorpholine, 2,4-diamino-1-(β-hydroxyethyloxy)benzene, 2-amino-3-hydroxypyridine, 5-amino-6-chloro-2-methylphenol, 1-methyl-2-hydroxy-4-β-hydroxyethylaminobenzene, 2-amino-4-hydroxyethylaminoanisole, hydroxyethyl-3,4-methylenedioxyaniline, 2-amino-5-ethylphenol and 1-hydroxy-3-aminobenzene, the addition salts thereof, and/or the solvates thereof, and/or solvates of the salts thereof, and mixtures thereof.

In general, the addition salts of the couplers that may be used include those chosen from addition salts with an acid, such as hydrochlorides, hydrobromides, sulfates, citrates, succinates, tartrates, lactates, tosylates, benzenesulfonates, phosphates and acetates, and the addition salts with a base such as sodium hydroxide, potassium hydroxide, aqueous ammonia, amines or alkanolamines.

Moreover, the solvates more particularly represent the hydrates of these couplers and/or the combination of these couplers with a linear or branched C1 to C4 alcohol such as methanol, ethanol, isopropanol or n-propanol. In certain embodiments, the solvates are hydrates.

Optionally, the couplers can be chosen from 6-hydroxybenzomorpholine, the addition salts thereof, the solvates thereof and/or the solvates of the salts thereof, hydroxyethyl-3,4-methylenedioxyaniline, the addition salts thereof, the solvates thereof and/or the solvates of the salts thereof, 2-amino-5-ethylphenol, the addition salts thereof, the solvates thereof and/or the solvates of the salts thereof, and mixtures thereof.

In a particular embodiment, the composition according to the invention is free of oxidation couplers chosen from resorcinol, 2-methylresorcinol, 4-chlororesorcinol, the addition salts thereof, the solvates thereof and the solvates of the salts thereof.

If present, the total amount of couplers in a hair color base composition for permanently or demi-permanently altering the color of hair can range from about 0.0001% to about 10%, such as from about 0.005% to about 7%, or from about 0.1% to about 4% by weight, relative to the total weight of the hair color base composition.

Optionally, compositions according to the disclosure comprise one or more direct dyes. The term “direct dye” means natural and/or synthetic dyes, other than oxidation dyes. These are dyes that will be deposited superficially onto the hair fiber. Direct dyes that can be used may be anionic (acidic), cationic (basic), or non-ionic (neutral).

The synthetic direct dyes are, for example, chosen from the dyes conventionally used for direct dyeing, and among which mention may be made of all the aromatic and/or non-aromatic dyes that are commonly used, such as nitrobenzene, azo, hydrazono, nitro(hetero)aryl, tri(hetero)arylmethine, (poly) methine, carbonyl, azine, porphyrin, metalloporphyrin, quinone and in particular anthraquinone, indolamine and phthalocyanine direct dyes, or combinations thereof.

Among the nitrobenzene direct dyes, mention may be made of: 1,4-diamino-2-nitrobenzene, 1-amino-2-nitro-4-β-hydroxyethylaminobenzene, 1-amino-2-nitro-4-bis(β-hydroxyethyl)aminobenzene, 1,4-bis(β-hydroxyethylamino)-2-nitrobenzene, 1-β-hydroxyethylamino-2-nitro-4-bis(β-hydroxyethylamino)benzene, 1-β-hydroxyethylamino-2-nitro-4-aminobenzene, 1-β-hydroxyethylamino-2-nitro-4-(ethyl) (β-hydroxyethyl)aminobenzene, 1-amino-3-methyl-4-β-hydroxyethylamino-6-nitrobenzene, 1-amino-2-nitro-4-β-hydroxyethylamino-5-chlorobenzene, 1,2-diamino-4-nitrobenzene, 1-amino-2-β-hydroxyethylamino-5-nitrobenzene, 1,2-bis(β-hydroxyethylamino)-4-nitrobenzene, 1-amino-2-tris(hydroxymethyl)methylamino-5-nitrobenzene, 1-hydroxy-2-amino-5-nitrobenzene, 1-hydroxy-2-amino-4-nitrobenzene, 1-hydroxy-3-nitro-4-aminobenzene, 1-hydroxy-2-amino-4,6-dinitrobenzene, 1-β-hydroxyethyloxy-2-β-hydroxyethylamino-5-nitrobenzene, 1-methoxy-2-β-hydroxyethylamino-5-nitrobenzene, 1-β-hydroxyethyloxy-3-methylamino-4-nitrobenzene, 1-β,γ-dihydroxypropyloxy-3-methylamino-4-nitrobenzene, 1-β-hydroxyethylamino-4-β, γ-dihydroxypropyloxy-2-nitrobenzene, 1-β,γ-dihydroxypropylamino-4-trifluoromethyl-2-nitrobenzene, 1-β-hydroxyethylamino-4-trifluoromethyl-2-nitrobenzene, 1-β-hydroxyethylamino-3-methyl-2-nitrobenzene, 1-β-aminoethylamino-5-methoxy-2-nitrobenzene, 1-hydroxy-2-chloro-6-ethylamino-4-nitrobenzene, 1-hydroxy-2-chloro-6-amino-4-nitrobenzene, 1-hydroxy-6-bis(β-hydroxyethyl)amino-3-nitrobenzene, 1-β-hydroxyethylamino-2-nitrobenzene, 1-hydroxy-4-β-hydroxyethylamino-3-nitrobenzene, or combinations thereof.

Among the azo direct dyes, mention may be made of: Basic Red 51, Basic Orange 31, Disperse Red 17, Acid Yellow 9, Acid Black 1, Basic Red 22, Basic Red 76, Basic Yellow 57, Acid Yellow 36, Acid Orange 7, Acid Red 33, Acid Red 35, Acid Yellow 23, Acid Orange 24, Disperse Black 9, Basic Brown 16, Basic Brown 17, or combinations thereof.

Among the hydrazono direct dyes, mention may be made of Basic Yellow 87.

Among the nitroaryl direct dyes, mention may be made of: HC Blue 2, HC Yellow 2, HC Red 3,4-hydroxypropylamino-3-nitrophenol, N,N′-bis(2-hydroxyethyl)-2-nitrophenylenediamine, or combinations thereof.

Among the triarylmethane direct dyes, mention may be made of: Basic Violet 1, Basic Violet 2, Basic Violet 3, Basic Violet 4, Basic Violet 14, Basic Blue 1, Basic Blue 7, Basic Blue 26, Basic Green 1, Basic Blue 77 (also known as HC Blue 15), Acid Blue 1; Acid Blue 3; Acid Blue 7, Acid Blue 9; Acid Violet 49; Acid Green 3; Acid Green 5; Acid Green 50, or combinations thereof.

Among the quinone direct dyes, mention may be made of: Disperse Red 15, Solvent Violet 13, Acid Violet 43, Disperse Violet 1, Disperse Violet 4, Disperse Blue 1, Disperse Violet 8, Disperse Blue 3, Disperse Red 11, Acid Blue 62, Disperse Blue 7, Basic Blue 22, Disperse Violet 15, Basic Blue 99, and also the following compounds: 1-N-methylmorpholiniumpropylamino-4-hydroxyanthraquinone, 1-aminopropylamino-4-methylaminoanthraquinone, 1-aminopropylaminoanthraquinone, 5-β-hydroxyethyl-1,4-diaminoanthraquinone, 2-aminoethylaminoanthraquinone, 1,4-bis(β,γ-dihydroxypropylamino) anthraquinone, Acid Blue 25, Acid Blue 43, Acid Blue 78, Acid Blue 129, Acid Blue 138, Acid Blue 140, Acid Blue 251, Acid Green 25, Acid Green 41, Acid Violet 42, Mordant Red 3, Acid Black 48, HC Blue 16, or combinations thereof.

Among the azine direct dyes, mention may be made of Basic Blue 17, Basic Red 2, or combinations thereof.

Among the indolamine direct dyes, mention may be made of: 2-β-hydroxyethylamino-5-[bis(3-4′-hydroxyethyl)amino]anilino-1,4-benzoquinone, 2-β-hydroxyethylamino-5-(2′-methoxy-4′-amino) anilino-1,4-benzoquinone, 3-N-(2′-chloro-4′-hydroxy)phenylacetylamino-6-methoxy-1,4-benzoquinoneimine, 3-N-(3′-chloro-4′-methylamino)phenylureido-6-methyl-1,4-benzoquinoneimine, 3-[4′-N-(ethylcarbamoylmethyl)amino]phenylureido-6-methyl-1,4-benzoquinoneimine, or combinations thereof.

The natural direct dyes are chosen, for example, from lawsone, juglone, indigo, leuco indigo, indirubin, isatin, hennotannic acid, alizarin, carthamine, morin, purpurin, carminic acid, kermesic acid, laccaic acid, purpurogallin, protocatechualdehyde, curcumin, spinulosin, apigenin, orceins, carotenoids, betanin, chlorophylls, chlorophyllines, monascus, polyphenols, ortho-diphenols, or combinations thereof.

Among the ortho-diphenols that are useful according to the invention, mention may be made of: catechin, quercetin, brazilin, hematin, haematoxylin, chlorogenic acid, caffeic acid, gallic acid, L-DOPA, cyanidin, (−)-epicatechin, (−)-epigallocatechin, (−)-epigallocatechin 3-gallate (EGCG), isoquercetin, pomiferin, esculetin, 6,7-dihydroxy-3-(3-hydroxy-2,4-dimethoxyphenyl) coumarin, santalin A and B, mangiferin, buten, maritimetin, sulfuretin, robtein, bethanidine, pericampylinone A, theaflavine, proanthocyanidin A2, proanthocyanidin B2, proanthocyanidin C1, procyanidins DP 4-8, tannic acid, purpurogallin, 5,6-dihydroxy-2-methyl-1,4-naphthoquinone, alizarin, wedelolactone and natural extracts containing same, or combinations thereof.

When the hair color base composition comprises one or more direct dyes, the total amount of direct dyes may range from about 0.001% to about 20%, such as from about 0.005% to about 15%, from about 0.01% to about 10%, from about 0.05% to about 5%, or from about 0.1% to about 3% by weight, relative to the weight of the hair color base composition.

### Solvents

Hair color base compositions according to the disclosure comprise a cosmetically acceptable solvent. The solvent may comprise water, non-aqueous solvents, or mixtures thereof.

In some embodiments, the solvent comprises, consists essentially of, or consists of water. The total amount of water in the hair color base compositions may vary depending on the type of composition and the desired consistency, viscosity, etc.

In certain embodiments, the hair color base composition comprises one or more non-aqueous solvents, other than or in addition to ingredients discussed above. For example, C1-C4 alcohols, organic solvents, fatty alcohols, fatty ethers, fatty esters, polyols other than those described above, glycols, vegetable oils, mineral oils, liposomes, laminar lipid materials, or any a mixture thereof. Non-limiting examples of solvents which may be used include alkane polyols such as 1,2,6-hexanetriol, trimethylolpropane, ethylene glycol, propylene glycol, diethylene glycol, hexylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, and 4-methyl-1,2-pentanediol; alkyl alcohols having 1 to 4 carbon atoms such as ethanol, methanol, butanol, propanol, and isopropanol (isopropyl alcohol); glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbitol, sorbitan, acetine, diacetone, triacetine, sulfolane, or mixtures thereof. At least in some embodiments, the hair color base compositions contain water and at least one, for example two or more, additional solvent chosen from non-aqueous solvents.

The solvent may be present in the hair color base composition in an amount ranging from about 20% to about 90% by weight, relative to the total weight of the composition, including all ranges and subranges therebetween. For example, in some embodiments, the total amount of solvent may range from about 35% to about 85%, such as from about 40% to about 80%, from about 45% to about 75%, or from about 50% to about 70% by weight, relative to the total weight of the hair color base composition.

In certain embodiments, the solvent is primarily or entirely comprised of water. For example, in various embodiments, the solvent comprises from about 80% to about 100%, such as from about 80% to about 99%, from about 80% to about 98%, from about 80% to about 97%, from about 80% to about 96%, from about 80% to about 95%, from about 80% to about 90%, from about 85% to about 100%, from about 85% to about 99%, from about 85% to about 98%, from about 85% to about 97%, from about 85% to about 96%, from about 85% to about 95%, from about 85% to about 90%, from about 90% to about 100%, from about 90% to about 99%, from about 90% to about 98%, from about 90% to about 97%, from about 90% to about 96%, or from about 90% to about 95% of water by weight, relative to the total weight of the solvent.

### Additional Components

Hair color base compositions according to the disclosure optionally comprise one or more additional components. The additional components described herein have been found to impart additional benefits to the hair, the hair color altering base composition, and/or the hair color altering composition, and/or to the process for altering the color of the hair, when present in various combinations and amounts. The additional components can be chosen from fatty alcohols, colloid agents, fatty compounds other than fatty alcohols, alkalizing agents, surfactants, conditioning polymers, amino acids other than glycine, and auxiliary agents.

Optionally, hair color base compositions according to the disclosure comprise at least one fatty alcohol. Suitable fatty alcohols include those having a fatty group with a chain having or at least five carbon atoms, such as, for example, from 8 to 50 carbon atoms, 8 to 40 carbon atoms, 8 to 30 carbon atoms, 8 to 22 carbon atoms, 12 to 22 carbon atoms, or 16 to 18 carbon atoms, including all ranges and subranges therebetween. In some instances, the fatty group of the fatty alcohols has a carbon chain of 10 to 20 carbon atoms or 10 to 18 carbon atoms. The fatty alcohols may be chosen from polyethylene glycol ethers, such as those having a fatty alcohol group with a carbon chain of 12 to 16 or 12 to 14 carbon atoms.

In some embodiments, the fatty alcohol portion is optionally hydrogenated (for example, stearyl, lauryl, cetyl, cetearyl); however, the fatty alcohol may contain one or more double bonds (for example, oleyl). Non-limiting examples of fatty alcohols include decyl alcohol, undecyl alcohol, dodecyl alcohol, myristyl alcohol, lauryl alcohol, cetyl alcohol, stearyl alcohol, cetearyl alcohol (cetyl alcohol and stearyl alcohol), isostearyl alcohol, isocetyl alcohol, behenyl alcohol, linalool, oleyl alcohol, cis-4-t-butylcyclohexanol, isotridecyl alcohol, myricyl alcohol, or mixtures thereof. In some cases, the fatty alcohols may be chosen from myristyl alcohol, lauryl alcohol, cetyl alcohol, stearyl alcohol, cetearyl alcohol, isostearyl alcohol, oleyl alcohol, isotridecyl alcohol, or mixtures of two or more thereof, for example cetearyl alcohol and oleyl alcohol.

The fatty alcohol may be saturated or unsaturated. In some embodiments, the hair color base compositions comprise at least one unsaturated fatty alcohol, and in other embodiments the hair color base compositions comprise at least one unsaturated fatty alcohol and at least one saturated fatty alcohol.

Exemplary saturated liquid fatty alcohols may be branched or straight-chain and optionally contain in their structure at least one aromatic or non-aromatic ring. In some instances, however, the fatty alcohols are acyclic. Non-limiting examples of liquid saturated fatty alcohols include cetyl alcohol, stearyl alcohol, cetearyl alcohol, octyldodecanol, isostearyl alcohol, and 2-hexyldecanol.

Exemplary unsaturated liquid fatty alcohol may include in their structure at least one double or triple bond. For example, the fatty alcohols may include several double bonds (such as 2 or 3 double bonds), which may be conjugated or non-conjugated. The unsaturated fatty alcohols can be linear or branched and may be acyclic or include in their structure at least one aromatic or non-aromatic ring. Liquid unsaturated fatty alcohols may include or be chosen from oleyl alcohol, linoleyl alcohol, linolenyl alcohol and undecylenyl alcohol.

If present, the total amount of fatty alcohols can vary, but typically ranges from about 1% to about 25%, such as from about 2% to about 22%, from about 3% to about 20%, from about 4% to about 18%, or from about 5% to about 16% by weight, relative to the total weight of the hair color base composition. For example, in various embodiments, the hair color base composition comprises a total amount of fatty alcohols ranging from about 2% to about 20%, from about 2% to about 18%, from about 2% to about 16%, from about 2% to about 15%, from about 2% to about 14%, from about 2% to about 13%, from about 2% to about 12%, from about 2% to about 11%, from about 2% to about 10%, from about 2% to about 9%, from about 3% to about 20%, from about 3% to about 18%, from about 3% to about 16%, from about 3% to about 15%, from about 3% to about 14%, from about 3% to about 13%, from about 3% to about 12%, from about 3% to about 11%, from about 3% to about 10%, from about 3% to about 9%, from about 4% to about 20%, from about 4% to about 18%, from about 4% to about 16%, from about 4% to about 15%, from about 4% to about 14%, from about 4% to about 13%, from about 4% to about 12%, from about 4% to about 11%, from about 4% to about 10%, from about 4% to about 9%, from about 5% to about 20%, from about 5% to about 18%, from about 5% to about 16%, from about 5% to about 15%, from about 5% to about 14%, from about 5% to about 13%, from about 5% to about 12%, from about 5% to about 11%, from about 5% to about 10%, from about 5% to about 9%, from about 6% to about 20%, from about 6% to about 18%, from about 6% to about 16%, from about 6% to about 15%, from about 6% to about 14%, from about 6% to about 13%, from about 6% to about 12%, from about 6% to about 11%, from about 6% to about 10%, from about 6% to about 9%, from about 7% to about 20%, from about 7% to about 18%, from about 7% to about 16%, from about 7% to about 15%, from about 7% to about 14%, from about 7% to about 13%, from about 7% to about 12%, from about 7% to about 11%, from about 7% to about 10%, from about 7% to about 9%, from about 8% to about 20%, from about 8% to about 18%, from about 8% to about 16%, from about 8% to about 15%, from about 8% to about 14%, from about 8% to about 13%, from about 8% to about 12%, from about 8% to about 11%, from about 8% to about 10%, from about 8% to about 9%, from about 9% to about 20%, from about 9% to about 18%, from about 9% to about 16%, from about 9% to about 15%, from about 9% to about 14%, from about 9% to about 13%, from about 9% to about 12%, from about 9% to about 11%, or from about 9% to about 10% by weight, relative to the total weight of the hair color base composition. For example, the hair color base composition may comprise at least one saturated and/or at least one unsaturated fatty alcohol, e.g. cetyl alcohol, stearyl alcohol, cetearyl alcohol, and/or oleyl alcohol, in any of the above amounts.

In some embodiments, however, the total amount of fatty alcohols can include any of the aforementioned ranges where the upper limit is not greater than about 15%, such as not greater than about 14%, not greater than about 13%, not greater than about 12%, not greater than about 11%, or not greater than about 10% by weight, relative to the total weight of the composition. By way of non-limiting example, the total amount of fatty alcohols may range from about 1% to about 15%, such as from about 2% to about 14%, from about 3% to about 13%, from about 4% to about 12%, or from about 5% to about 11% by weight, relative to the total weight of the hair color base composition.

In some embodiments, a hair color base composition according to the disclosure comprises at least two fatty alcohols, e.g., at least one saturated and at least one unsaturated fatty alcohol. By way of example, the compositions may comprise at least one saturated fatty alcohol chosen from cetyl alcohol, stearyl alcohol, and/or cetearyl alcohol, and at least one unsaturated fatty alcohol which can include oleyl alcohol.

Hair color base compositions may also comprise one or more colloid agents. Surprisingly, by including such compounds in certain amounts, the fatty alcohol content can be lowered while maintaining stability and integrity of the composition.

Useful and non-limiting examples of colloid agents include hydrophilic polymers, such as, for example, cellulose and its derivatives, e.g. carboxymethylcellulose, hydroxypropylcellulose, methylcellulose, hydroxyethylcellulose, and hydroxypropylmethylcellulose, as well as modified celluloses, such as those modified by grafting of the alkyl group. For example, hydroxyethylcellulose and/or cetyl hydroxyethyl cellulose may be chosen. Other useful and non-limiting examples of colloid agents include seaweed extracts such as agar agar, carrageenans, and alginates; seed extracts such as carob gum, guar gum, and modified guar gums, especially those modified by grafting of the alkyl group; plant exudates such as gum arabic, karaya gum, gum tragacanth, and gatty gum; synthetic water-soluble gelling polymers such as crosslinked polyacrylic acids; fruit extracts such as pectins; and microorganism exudates such as xanthan gum.

The total amount of colloid agents can vary, and may range, for example, from about 0.01% to about 2%, such as from about 0.05% to about 1.5%, from about 0.1% to about 1.25%, from about 0.2% to about 1%, from about 0.25% to about 0.75%, or from about 0.3% to about 0.7% by weight, relative to the total weight of the hair color base composition. In some embodiments, the total amount of colloid agents ranges from about 0.01% to about 1%, such as from about 0.05% to about 0.75%, from about 0.1% to about 0.5%, or from about 0.2% to about 0.4% by weight, relative to the total weight of the hair color base composition.

In other embodiments, however, the total amount of colloid agents may range from about 0.1% to about 1.5%, such as from about 0.2% to about 1%, from about 0.3% to about 0.9%, from about 0.2% to about 0.8%, or from about 0.45% to about 0.75% by weight, relative to the total weight of the hair color base composition. It may, in some embodiments when such amounts of colloid agent are chosen, be possible to use lower amounts of fatty alcohols but without interfering with the stability and integrity of the hair color base composition. Thus, in some embodiments, the total amount of fatty alcohols can include any of the aforementioned ranges described for fatty alcohols wherein the upper limit is not greater than about 15%, such as not greater than about 14%, not greater than about 13%, not greater than about 12%, not greater than about 11%, or not greater than about 10% by weight, relative to the total weight of the hair color base composition.

By way of non-limiting example, hair color base compositions may comprise one or more colloid agents, e.g. hydroxyethyl cellulose and/or cetyl hydroxyethyl cellulose, in a total amount ranging from about 0.1% to about 1.5%, from about 0.2% to about 1%, from about 0.3% to about 0.9%, from about 0.2% to about 0.8%, or from about 0.45% to about 0.75% by weight, relative to the total weight of the hair color base composition, and one or more fatty alcohols, e.g., at least one saturated and/or at least one unsaturated fatty alcohol, for example chosen from cetyl alcohol, stearyl alcohol, cetearyl alcohol, and/or oleyl alcohol, wherein the total amount of fatty alcohols is not greater than about 15% or not greater than about 12%, for example ranges from about 1% to about 15%, from about 3% to about 14%, from about 5% to about 13%, from about 6% to about 12%, from about 7% to about 11%, or from about 8% to about 10% by weight, relative to the total weight of the hair color base composition.

Hair color base compositions according to the disclosure may optionally further comprise at least one fatty compound other than fatty alcohols. By way of non-limiting example, fatty compounds other than fatty alcohols may be chosen from fatty acids and esters of fatty acids, oils of animal, vegetable, or mineral origin, silicone oils, lower alkanes, or combinations of two or more thereof.

Suitable fatty acids that can be included in the base compositions may have about 10 to about 30 carbon atoms, from about 12 to about 22 carbon atoms, or from about 16 to about 22 carbon atoms. These fatty acids can be straight or branched chain acids and can be saturated or unsaturated. Also included are diacids, triacids, and other multiple acids which meet the carbon number requirement herein. Further included herein are salts of these fatty acids. Nonlimiting examples of fatty acids include myristic acid, lauric acid, palmitic acid, stearic acid, behenic acid, arachidonic acid, oleic acid, isostearic acid, sebacic acid, or mixtures thereof. In some embodiments, the fatty acids may comprise myristic acid, palmitic acid, stearic acid, or mixtures thereof.

In various embodiments, hair color base compositions according to the disclosure may further comprise one or more vegetable oils. As used herein, the term “vegetable oils” refers to oils derived from a plant, for example, oils from seeds or fruits.

In some embodiments, suitable vegetable oils are chosen from hydrocarbon-based oils of plant origin and/or vegetable origin. Non-limiting examples of plant based or vegetable based oils include acai oil, almond oil, aloe vera oil, andiroba oil, annatto oil, avocado oil, babassu oil, borage oil, brazil nut oil, buriti oil, camelina oil, coffee oil, copaiba oil, emu oil, passion fruit oil, almond oil, castor oil, coconut oil, grapeseed oil, jojoba oil, macadamia nut oil, rose hip oil, ajwain oil, angelic root oil, anise oil, aragan oil, asafetida, balsam oil, basil oil, bay oil, bergamot oil, black pepper essential oil, buchu oil, birch oil, camphor, cannabis oil, caraway oil, cardamom seed oil, carrot seed oil, chamomile oil, calamus root oil, cinnamon oil, citronella oil, clary sage, clove leaf oil, coffee, coriander, costmary oil, cranberry seed oil, cubeb, cumin oil, cypress, cypriol, curry leaf, davana oil, dill oil, elecampane, eucalyptus oil, fennel seed oil, fenugreek oil, fir, frankincense oil, galangal, geranium oil, ginger oil, goldenrod, grapefruit oil, grapeseed oil, henna oil, helichrysum, horseradish oil, hyssop, Idaho tansy, jasmine oil, juniper berry oil, lavender oil, lemon oil, lemongrass, marjoram, melaleuca, lemon balm oil, mountain savory, mugwort oil, mustard oil, myrrh oil, myrtle, neem tree oil, neroli, nutmeg, orange oil, oregano oil, orris oil, palo santo, parsley oil, patchouli oil, perilla oil, pennyroyal oil, peppermint oil, petitgrain, pine oil, plum oil, ravensara, red cedar, roman chamomile, rose oil, rosehip oil, rosemary oil, rosewood oil, sandalwood oil, sassafras oil, savory oil, schisandra oil, spikenard, spruce, star anise oil, tangerine, tarragon oil, tea tree oil, thyme oil, tsuga oil, turmeric, valerian, vetiver oil, western red cedar, wintergreen, yarrow oil, ylang-ylang, and zedoary oil.

In some cases, the vegetable oils may be glyceride triesters, which are generally triesters of fatty acids and of glycerol, the fatty acids of which can have varied chain lengths from C4 to C24, it being possible for these chains to be saturated or unsaturated and linear or branched; these oils are in particular wheat germ oil, sunflower oil, grape seed oil, sesame oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin seed oil, marrow oil, blackcurrant oil, evening primrose oil, millet oil, barley oil, quinoa oil, rye oil, safflower oil, candlenut oil, passionflower oil, and musk rose oil.

In some embodiments, a hair color base composition according to the present disclosure includes at least one oil chosen from saturated or unsaturated plant oils having less than 16 carbon atoms, for example, coconut oil, Ricinus communis (castor) seed oil, Butyrospermum parkii (shea) butter, palmitic acid, jojoba oil, or a mixture thereof.

If present, the total amount of fatty compounds other than fatty alcohols typically ranges from about 0.5% to about 20%, including all subranges therebetween, such as from about 0.5% to about 15%, from about 0.5% to about 12%, from about 0.5% to about 10%, from about 0.5% to about 8%, from about 0.5% to about 6%, from about 0.5% to about 4%, from about 0.5% to about 3%, from about 1% to about 20%, from about 1% to about 15%, from about 1% to about 12%, from about 1% to about 10%, from about 1% to about 8%, from about 1% to about 6%, from about 1% to about 4%, from about 2% to about 20%, from about 2% to about 15%, from about 2% to about 12%, from about 2% to about 10%, from about 2% to about 8%, from about 2% to about 6%, or from about 2% to about 4% by weight, relative to the total weight of the hair color base composition.

Hair color base compositions according to the disclosure may optionally comprise at least one alkalizing agent. The alkalizing agents may be chosen from organic alkalizing agents and/or mineral alkalizing agents. In some embodiments, the hair color base compositions comprise only organic alkalizing agents. In other embodiments, the hair color base compositions comprise only mineral alkalizing agents. In still other embodiments, the hair color base compositions a combination of organic and mineral alkalizing agents.

As non-limiting examples, suitable organic alkalizing agents may be chosen from alkanolamines. In various embodiment, suitable alkanolamines may be chosen from mono-, di- or trialkylamines, comprising one to three identical or different C1-C4 hydroxyalkyl radicals, ethylamines, ethyleneamines, quinoline, aniline and cyclic amines, such as pyrroline, pyrrole, pyrrolidine, imidazole, imidazolidine, imidazolidinone, morpholine, pyridine, piperidine, pyrimidine, piperazine, triazine and derivatives thereof. In some embodiments, alkanolamine alkalizing agents that may be mentioned include but not limited to monoethanolamine (also known as ethanolamine or MEA), diethanolamine, triethanolamine, monoisopropanolamine, diisopropanolamine, N-dimethylaminoethanolamine, 2-amino-2-methyl-1-propanol, triisopropanolamine, 2-amino-2-methyl-1,3-propanediol, 3-amino-1,2-propanediol, 3-dimethylamino-1,2-propanediol, 2-amino-2-methyl-1-propanol, and tris(hydroxymethylamino) methane. In some embodiments, the compositions comprise at least one alkalizing agent chosen from alkanolamines, such as monoethanolamine. Optionally, the organic alkalizing agent may comprise, consist essentially of, or consist of alkanolamines, for example monoethanolamine.

As non-limiting examples, suitable mineral alkalizing agents may be chosen from ammonia, ammonium carbonates, sodium carbonates, potassium carbonates, ammonium bicarbonates, sodium bicarbonates, potassium bicarbonates, ammonium hydroxides, sodium hydroxides, potassium hydroxides, salts thereof, hydrates thereof, or mixtures thereof. In some embodiments, the hair color base compositions comprise ammonia and/or ammonium hydroxide. Optionally, the mineral alkalizing agent may consist essentially of or consist of ammonia and/or ammonium hydroxide.

In some embodiments, a hair color base composition disclosed herein comprises at least one organic alkalizing agent and at least one mineral alkalizing agent. In some embodiments, the at least one alkalizing agent comprises, consists essentially of or consists of ammonia or ammonium hydroxide, alkanolamines, salts thereof, hydrates thereof, or combinations thereof. In at least one embodiment, a hair color base composition disclosed herein comprises both monoethanolamine and ammonium hydroxide.

In some embodiments, alkalizing agents present in a hair color base composition may be only chosen from organic alkalizing agents, and the hair color base composition is free or substantially free of mineral alkalizing agents, such as ammonia and/or ammonium hydroxide. In this case, the hair color base composition may comprise less than about 0.5%, less than about 0.4%, less than about 0.3%, less than about 0.2%, less than about 0.1%, or less than about 0.05% of mineral alkalizing agents. In certain embodiments, the hair color base compositions disclosed herein comprise one or more organic alkalizing agents and are free or substantially free of ammonia and/or ammonium-based compounds.

In various embodiments, the amount of organic or mineral alkalizing agents may independently range from about 0.1% to about 10%, such as from about 0.1% to about 9%, from about 0.1% to about 8%, from about 0.1% to about 7%, from about 0.1% to about 6%, from about 0.1% to about 5%, from about 0.1% to about 4%, from about 0.1% to about 3%, from about 0.1% to about 2%, from about 0.1% to about 1%, from about 0.5% to about 10%, from about 0.5% to about 9%, from about 0.5% to about 8%, from about 0.5% to about 7%, from about 0.5% to about 6%, from about 0.5% to about 5%, from about 0.5% to about 4%, from about 0.5% to about 3%, from about 0.5% to about 2%, from about 0.5% to about 1%, from about 0.75% to about 10%, from about 0.75% to about 9%, from about 0.75% to about 8%, from about 0.75% to about 7%, from about 0.75% to about 6%, from about 0.75% to about 5%, from about 0.75% to about 4%, from about 0.75% to about 3%, from about 0.75% to about 2%, from about 0.75% to about 1%, from about 1% to about 10%, from about 1% to about 9%, from about 1% to about 8%, from about 1% to about 7%, from about 1% to about 6%, from about 1% to about 5%, from about 1% to about 4%, from about 1% to about 3%, from about 1% to about 2%, from about 2% to about 10%, from about 2% to about 9%, from about 2% to about 8%, from about 2% to about 7%, from about 2% to about 6%, from about 2% to about 5%, from about 2% to about 4%, from about 2% to about 3%, from about 3% to about 10%, from about 3% to about 9%, from about 3% to about 8%, from about 3% to about 7%, from about 3% to about 6%, from about 3% to about 5%, from about 3% to about 4%, from about 4% to about 10%, from about 4% to about 9%, from about 4% to about 8%, from about 4% to about 7%, from about 4% to about 6%, from about 4% to about 5%, from about 5% to about 10%, from about 5% to about 9%, from about 5% to about 8%, from about 5% to about 7%, or from about 5% to about 6% by weight, based on the total weight of the hair color base composition.

For example, in some embodiments, the alkalizing agents may comprise at least one organic alkalizing agent and at least one mineral alkalizing agent, wherein the at least one organic alkalizing agent is present in an amount ranging from about 0.1% to about 5%, such as from about 0.2% to about 4%, from about 0.3% to about 3%, from about 0.4% to about 2%, or from about 0.5% to about 1.5% by weight, relative to the total weight of the hair color base composition; and the at least one mineral alkalizing agent is present in an amount ranging from about 0.5% to about 10%, such as from about 1% to about 8%, from about 2% to about 6%, or from about 3% to about 5% by weight, relative to the total weight of the hair color base composition. For instance, the hair color base composition may comprise at least one alkanolamine, e.g. monoethanolamine, in combination with ammonia and/or ammonium hydroxide, in the above-mentioned ranges.

As a further example, in some embodiments, the alkalizing agent consists of or consists essentially of organic alkalizing agents, e.g. alkanolamines, wherein the total amount of organic alkalizing agents ranges from about 0.5% to about 10%, such as from about 1% to about 9%, from about 2% to about 8%, or from about 3% to about 7% by weight, relative to the total weight of the hair color base composition. In such embodiments, the compositions may comprise less than about 0.5%, less than about 0.4%, less than about 0.3%, less than about 0.2%, less than about 0.1%, less than about 0.05%, or less than about 0.01% of mineral alkalizing agents, e.g. ammonia and/or ammonium-based compounds.

In various embodiments, the hair color base compositions comprise one or more surfactants. For example, the compositions may comprise one or more nonionic surfactants and/or anionic surfactants, and in some embodiments the compositions may comprise mixtures of surfactants having the same or different ionicities.

Useful and non-limiting examples of nonionic surfactants include, for example, polyoxyalkylenated fatty alcohols, alkyl polyglucosides, oxyalkylenated glycerol esters, oxyalkylenated fatty acid esters of sorbitan (e.g. polysorbate 60), polyoxyalkylenated (e.g. polyoxyethylenated and/or polyoxypropylenated) fatty acid esters optionally in combination with a fatty acid ester of glycerol, such as the PEG-100 stearate/glyceryl stearate mixture, oxyalkylenated sugar esters, or mixtures thereof.

Non-limiting examples of polyoxyalkylenated fatty alcohols that can be chosen include ceteareth-2, ceteareth-3, ceteareth-4, ceteareth-5, ceteareth-6, ceteareth-7, ceteareth-8, ceteareth-9, ceteareth-10, ceteareth-11, ceteareth-12, ceteareth-13, ceteareth-14, ceteareth-15, ceteareth-16, ceteareth-17, ceteareth-18, ceteareth-20, ceteareth-22, ceteareth-23, ceteareth-24, ceteareth-25, ceteareth-27, ceteareth-28, ceteareth-29, ceteareth-30, ceteareth-33, ceteareth-34, ceteareth-40, ceteareth-50, ceteareth-55, ceteareth-60, ceteareth-80, ceteareth-100, laureth-1, laureth-2, laureth-3, laureth-4, laureth-5, laureth-6, laureth-7, laureth-8, laureth-9, laureth-10, laureth-11, laureth-12, laureth-13, laureth-14, laureth-15, laureth-16, laureth-20, laureth-23, laureth-25, laureth-30, laureth-40, deceth-3, deceth-5, oleth-5, oleth-30, steareth-2, steareth-10, steareth-20, steareth-100, cetylsteareth-12, ceteareth-5, ceteareth-5, polyglyceryl 4-lauryl ether, polyglyceryl 4-oleyl ether, polyglyceryl 2-oleyl ether, polyglyceryl 2-cetyl ether, polyglyceryl 6-cetyl ether, polyglyceryl 6-oleylcetyl ether, polyglyceryl 6-octadecyl ether, C9-C11 pareth-3, C9-C11 pareth-6, C11-C15 pareth-3, C11-C15 pareth-5, C11-C15 pareth-12, C, 11-C15 pareth-20, C12-C15 pareth-9, C12-C15 pareth-12, and C22-C24 pareth-33. In some embodiments, the surfactant is chosen from polyoxyalkylenated fatty alcohols, for example laureth-12 and/or oleth-30.

Alkyl polyglucosides that can be used include, for example those containing an alkyl group including from 6 to 30 carbon atoms, for example from 8 to 16 carbon atoms, and containing a glucoside group, e.g. comprising from 1.2 to 3 glucoside units. The alkylpolyglucosides may be chosen, for example, from decylglycoside, caprylyl/caproyl glucoside, laurylglucoside, cocoyl glucoside, caprylyl glucoside, or mixtures thereof.

The oxyalkylenated glycerol esters that can be used include, for example, polyoxyethylenated derivatives of esters of glycerol and of a fatty acid and of their hydrogenated derivatives. These oxyalkylenated glycerol esters may be chosen, for example, from glyceryl esters of fatty acids which are hydrogenated and oxyethylated, such as PEG-200 hydrogenated glyceryl palmate, oxyethylated glyceryl cocoates, PEG-30 glyceryl cocoate, oxyethylated glyceryl stearates, or mixtures thereof.

The oxyalkylenated sugar esters can be chosen from polyethylene glycol ethers of fatty acid and sugar esters. These oxyalkylenated sugar esters may be chosen, for example, from oxyethylated glucose esters, such as PEG-120 methyl glucose dioleate.

As useful and non-limiting examples of anionic surfactants, mention may be made of the polyoxyalkylenated alkyl ether carboxylic acids, polyoxyalkylenated alkylaryl ether carboxylic acids, polyoxyalkylenated alkylamido ether carboxylic acids, in particular those including 2 to 50 ethylene oxide groups, alkyl D-galactoside uronic acids, acyl sarcosinates, acyl glutamates and alkylpolyglycoside carboxylic esters.

In at least some embodiments, the hair color base compositions comprise at least one nonionic surfactant and at least one anionic surfactant.

The total amount of surfactants may vary, and may range, for example, from about 1% to about 20% by weight, such as from about 2% to about 18%, from about 3% to about 16%, from about 4% to about 14%, from about 5% to about 12%, or from about 6% to about 11% by weight, relative to the total weight of the hair color base composition.

Optionally, hair color base compositions according to the disclosure may further comprise at least one conditioning polymer. Useful conditioning polymers include, for example, cationic polymers. In some embodiments, hair color base compositions comprise at least one polyquaternium compound.

For example, non-limiting examples of cationic polymers include copolymers of 1-vinyl-2-pyrrolidine and 1-vinyl-3-methyl-imidazolium salt (e.g., chloride salt) (referred to as Polyquaternium-16); copolymers of 1-vinyl-2-pyrrolidine and dimethylaminoethyl methacrylate (referred to as Polyquaternium-11); cationic diallyl quaternary ammonium-containing polymer including, for example, dimethyldiallylammonium chloride homopolymer and copolymers of acrylamide and dimethyldiallylammonium chloride (Polyquaternium-6 and Polyquaternium-7); polysaccharide polymers, such as cationic cellulose derivatives and cationic starch derivatives. Cationic cellulose is available as salts of hydroxyethyl cellulose reacted with trimethyl ammonium substituted epoxide (referred to as Polyquaternium-10). Another type of cationic cellulose includes the polymeric quaternary ammonium salts of hydroxyethyl cellulose reacted with lauryl dimethyl ammonium-substituted epoxide (referred to as Polyquaternium-24). Additionally, or alternatively, the cationic conditioning polymers may include or be chosen from cationic guar gum derivatives, such as guar hydroxypropyltrimonium chloride.

In some embodiments, cationic conditioning polymers include cationic polysaccharide polymers, such as cationic cellulose, cationic starch, and cationic guar gum. In the context of the instant disclosure cationic polysaccharide polymers include cationic polysaccharides and polysaccharide derivatives (e.g., derivatized to be cationic), for example, resulting in cationic cellulose (cellulose derivatized to be cationic), cationic starch (derivatized to be cationic), cationic guar (guar derivatized to be cationic). In further embodiments, cationic conditioning polymers include cationic guars such as guar hydroxypropyltrimonium chloride and hydroxypropyl guar hydroxypropyltrimonium chloride, or cationic starches such as starch hydroxypropyltrimonium chloride and hydroxypropyl oxidized starch PG trimonium chloride.

In some embodiments, quaternized polymeric cationic polymers are particularly useful, such as quaternary nitrogen polymers prepared by the polymerization of a doalkyldialkylammonium salt or copolymer thereof in which the alkyl group contains from 1 to about 18 carbon atoms, such as where the alkyl group is methyl or ethyl. For example, cationic homopolymers and copolymers of polydiallyldimethylammonium chloride may be chosen. For example, Polyquaternium-1, Polyquaternium-2, Polyquaternium-4, Polyquaternium-5 Polyquaternium-6, Polyquaternium-7, Polyquaternium-8, Polyquaternium-9, Polyquaternium-10, Polyquaternium-11, Polyquaternium-12, Polyquaternium-13, Polyquaternium-14, Polyquaternium-15, Polyquaternium-16, Polyquaternium-17, Polyquaternium-18, Polyquaternium-19, Polyquaternium-20, Polyquaternium-22, Polyquaternium-24, Polyquaternium-27, Polyquaternium-28, Polyquaternium-29, Polyquaternium-30, Polyquaternium-31, Polyquaternium-32, Polyquaternium-33, Polyquaternium-34, Polyquaternium-35, Polyquaternium-36, Polyquaternium-37, Polyquaternium-39, Polyquaternium-42, Polyquaternium-43, Polyquaternium-44, Polyquaternium-45, Polyquaternium-46, Polyquaternium-47, or a combination of two or more thereof, can be chosen. In at least some embodiments, the hair color base compositions comprise Polyquaternium-22. In further embodiments, the hair color base compositions are free or substantially free of Polyquaternium-6.

If present, the total amount of cationic polymers can range from about 0.01% to about 10%, such as from about 0.1% to about 5%, about 0.1% to about 3%, about 0.1% to about 2%, about 0.1% to about 1%, about 0.25% to about 5%, about 0.25% to about 3%, about 0.25% to about 2%, about 0.25% to about 1%, about 0.5% to about 5%, about 0.5% to about 3%, about 0.5% to about 2%, about 0.5% to about 1%, about 0.75% to about 5%, about 0.75% to about 3%, about 0.75% to about 2%, or about 0.75% to about 1% by weight, relative to the total weight of the hair base composition.

Optionally, the hair color base compositions may comprise one or more auxiliary components. Hair color base compositions according to the disclosure may optionally comprise one or more additional components suitable for use in cosmetic compositions, and in particular suitable for altering the color of hair. Such auxiliary components may include, for example, pH adjusters, thickeners other than colloid agents described above, vitamins, ceramides, preservatives, fillers, structuring agents, propellants, shine agents, antioxidants, penetrants, sequestrants, fragrances, buffers, dispersants, plant extracts, opacifiers, sunscreen agents, chelants, dyes or pigments for coloring the composition, and antistatic agents. Such optional auxiliary components will be chosen so as to minimize any detrimental effect to the advantages of the hair color base compositions described herein.

Optional auxiliary components may be present in an amount ranging up to about 15%, such as from about 0.001% to about 10%, from about 0.01% to about 5%, or from about 0.1% to about 3% by weight, relative to the total weight of the hair color base composition.

Typically, hair color base compositions according to the disclosure have a pH of greater than 7, such as greater than about 8, greater than about 9, or greater than about 10, ranging up to about 13. For example, the pH of the hair color base composition may range from about 9 to about 13, from about 9 to about 12, from about 9.5 to about 12, from about 10 to about 12, or from about 10.5 to about 11.5. In various embodiments, the hair color base composition has a pH of about 9.5, about 10, about 10.5, about 11, about 11.5, or about 12, including all ranges and subranges thereof.

Hair color base compositions according to the disclosure are typically in the form of a cream, paste, or lotion, but may also be in the form of a serum, a gel, a gel cream, or the like. In some embodiments, the hair color base compositions may be a rinse-off hair dye product, that can be used to lift hair color and/or provide desired color to the hair after being mixed with a developer composition comprising one or more oxidizing agents. Hair color base compositions according to the disclosure are generally stable and can be stored at room temperature for at least 24 months.

### Developer Compositions

Hair color base compositions disclosed herein can be mixed with a developer composition (also referred to as a “developer” or “oxidizing composition”) that comprises one or more oxidizing agents, resulting in mixtures (also referred to herein as “hair color altering compositions”) that are suitable for applying onto the hair for altering the color of the hair, such as, for example, by lifting the color of hair and/or providing color to the hair, and at the same time, providing benefits such as maintaining and/or enhancing the strength and/or elasticity of the hair, and reducing damage to hair due to chemical treatment caused by compositions and processes for altering the color of the hair.

Developer compositions can be mixed with hair color base compositions according to the disclosure to prepare a composition for altering the color of the hair. Typically, a developer composition comprises at least one oxidizing agent and a cosmetically suitable carrier. Developer compositions that can be used to prepare a hair color altering composition according to the disclosure are not limited, and can include, for example, any commercial developer composition as long as it includes at least one oxidizing agent.

In various embodiments, a suitable oxidizing agent may be chosen, for example, from peroxides, such as hydrogen peroxide and urea peroxide; persalts, such as persulfates, perborates, percarbonates, peroxygenated salts, etc., alkali metal bromates, alkali metal ferricyanides, alkali metal carbonates, or a mixture thereof. An oxidizing agent may also be chosen from one or more oxidation-reduction enzymes, such as laccases, peroxidases, and 2-electron oxidoreductases (such as uricase), optionally in the presence of their respective donor or cofactor.

In one embodiment, a suitable oxidizing agent may be chosen from persulfates and/or monopersulfates such as, for example, potassium persulfate, sodium persulfate, ammonium persulfate, as well as mixtures thereof. In one embodiment, the oxidizing agents in the present disclosure are chosen from hydrogen peroxide, potassium persulfate, sodium persulfate, and mixtures thereof.

In certain embodiments, hydrogen peroxide is used as an oxidizing agent. In certain situations, hydrogen peroxide may be present in an aqueous developer whose titer may range from 1 to 40 volumes, such as from 5 to 40 volumes, from 5 to 30 volumes, or from 5 to 20 volumes. In certain embodiments, the oxidizing composition is a 10V, 20V, 30V, or 40V hydrogen peroxide developer composition.

The total amount of oxidizing agents in the developer composition may, in various embodiments, range from about 0.05% to about 50% by weight, such as from about 0.1% to about 30% by weight, from about 0.1% to about 20% by weight, about 1% to about 20%, about 1% to about 15%, about 1% to about 12%, about 3% to about 20%, about 3% to about 15%, about 3% to about 12%, about 5% to about 20%, about 5% to about 15%, about 5% to about 12%, about 7% to about 20%, about 7% to about 15%, about 7% to about 12%, about 9% to about 20%, about 9% to about 15%, or about 9% to about 12% by weight, based on the total weight of the developer composition.

The developer composition may contain at least one solvent, chosen from, for example water, organic solvents, or mixtures thereof. Suitable organic solvents for use in the developer composition, alone or in mixture with water, include but are not limited to ethanol, isopropyl alcohol, propanol, benzyl alcohol, phenyl ethyl alcohol, glycols and glycol ethers, such as propylene glycol, hexylene glycol, ethylene glycol monomethyl, monoethyl or monobutyl ether, propylene glycol and its ethers, such as propylene glycol monomethyl ether, butylene glycol, dipropylene glycol, diethylene glycol alkyl ethers, such as diethylene glycol monoethyl ether and monobutyl ether, ethylene glycol, propylene glycol, butylene glycol, hexylene glycol, propane diol, glycerin, hydrocarbons such as straight chain hydrocarbons, mineral oil, polybutene, hydrogenated polyisobutene, hydrogenated polydecene, polydecene, squalane, petrolatum, isoparaffins, or mixtures thereof.

The organic solvents for use in the developer compositions can be volatile or non-volatile compounds. The organic solvent may, for example, be present in an amount ranging from about 0.5% to about 70% by weight, such as from about 2% to about 60% by weight, such as from about 5 to about 50% by weight, relative to the total weight of the developer composition.

The developer compositions are typically aqueous, but may also be in the form of a powder, gel, liquid, foam, lotion, cream, mousse, or emulsion. For instance, in certain embodiments the developer composition is aqueous and is in the form of a liquid, cream, or emulsion. In alternative embodiments, the developer composition may be anhydrous or substantially anhydrous. The term “substantially anhydrous” means that the developer composition is either completely free of water or contains no appreciable amount of water, for example, no more than 5% by weight, or no more than 2% by weight, or no more than 1% by weight, based on the weight of the developer composition. It should be noted that this refers for example to bound water, such as the water of crystallization of the salts or traces of water absorbed by the raw materials used in the preparation of the compositions according to embodiments of the disclosure. When the developer composition is substantially anhydrous, the developer composition may comprise at least one solvent chosen from organic solvents described above.

The developer compositions may optionally include other components typically used in developer compositions, such as, for example, rheology-modifying agents, chelants, fatty substances, ceramides, pH adjusting agents, preservatives, fragrances, surfactants, etc.

The pH of the developer composition is typically below 7, such as below about 6, below about 5, or below about 4. For example, the pH of the developer composition may range from about 1 to about 5, such as from about 1.5 to about 4 or from about 2 to about 3.

### Hair Color Altering Compositions

In non-limiting embodiments, the disclosure also provides hair color altering compositions, which comprise components described above for hair color base compositions and at least one oxidizing agent. The hair color altering compositions are typically prepared by mixing a hair color base composition disclosed herein with a developer composition, for example at or near the time of use.

According to various embodiments, a hair color base composition disclosed herein can be mixed with a developer composition in a weight ratio of hair color base composition to developer that ranges from about 1:5 to about 5:1, such as from about 1:4 to about 4:1, about 1:3 to about 3:1, or about 1:2 to about 2:1, or that is about 5:1, about 4:1, about 3:1, about 2:1, about 1.5:1, about 1:1, about 1:1.5, about 1:2, about 1:3, about 1:4, or about 1:5. It will be understood that, depending on the mix ratio, the amounts of components described herein for the hair color base and developer compositions will be reduced in the final hair color altering composition. For example, if a hair color base composition and developer composition are mixed at a 1:1 ratio, amounts disclosed herein for each will be present at 50% in the final mixture.

In some embodiments, the hair color altering compositions are free or essentially free of ammonia, salts thereof, and/or hydrates thereof. However, in at least some other embodiments, the compositions disclosed herein may comprise ammonia, salts thereof, and/or hydrates thereof.

The pH of the hair color altering composition resulting from mixing together the hair color base composition and developer composition is typically greater than 7, which can be adjusted to the desired value using acidifying/alkalizing agents that are well known in the art. In certain embodiments, the pH of the hair color altering composition is from about 7 to about 12, such as from about 8 to about 12, from about 9 to about 12, from about 7 to about 11, from about 8 to about 11, from about 9 to about 11. In some embodiments, the hair color altering composition has a pH ranging from about 10 to about 11.

### II. Kits

The present disclosure also relates to kits comprising the hair color base compositions described herein. The kits in various embodiments may comprise at least one container or compartment suitable for containing and/or dispensing the hair color base compositions, and optionally at least one second container or compartment. The optional second container or compartment may comprise, for example, a developer composition. In some other embodiments, a kit may comprise a first container containing a hair color base composition according to the disclosure, a second container comprising a developer comprising at least one oxidizing agent described above, and optionally, an additional separate container comprising a cosmetic composition, for example, a shampoo, a conditioner, a hair mask, a hair styling composition, or a hair treatment composition other than a developer or a base described above. The hair color base composition and the developer composition contained in separate containers may be mixed prior to the application onto hair.

In further embodiments, a kit according to the disclosure comprises a first container or compartment comprising a composition comprising at least one hair color altering agent, for example at least one oxidation dye compound or at least one direct dye as described above, and at least a second container or compartment comprising one or more of a carboxylic acid and/or a salt thereof, and glycine and/or a salt thereof. In such embodiments, it may be possible to provide a hair color base composition in a deconstructed manner, wherein the kit further provides instructions for mixing or combining contents of multiple compartments or containers in order to prepare a hair color base composition and/or a hair color altering composition as described herein, e.g. at or near the time of use.

### III. Methods

Hair color altering compositions described herein can effectively alter the color of hair, while at the same time surprisingly providing fiber strength and/or elasticity, and/or minimizing or reducing damage to hair caused by the hair color altering treatment. Therefore, another embodiment of the disclosure relates to methods of using any of the compositions disclosed herein for altering the color of the hair, including lifting the color of hair and/or providing a desired color to the hair, as well as to improving fiber integrity of the hair and/or minimizing or reducing damage to the hair caused by chemical treatment. In other embodiments, the methods relate to treating hair with compositions described herein to impart fiber integrity benefits such as strength and/or elasticity to hair.

In various embodiments, the methods disclosed herein typically comprise applying to hair an effective amount of a hair color altering composition disclosed herein. The hair color altering composition is generally obtained at the time or near the time of use by mixing a hair color base composition disclosed herein and a developer composition comprising at least one oxidizing agent. In some embodiments, the methods may further include mixing a hair color base composition disclosed herein and a developer composition described herein at the time or near the time of use.

In various embodiments, before application of the compositions disclosed here, the hair may be bleached, shampooed, and/or rinsed. In some embodiments, before application of the hair color altering composition to the hair, the hair may be first cleansed with a commercially available shampoo or be rinsed with water. The hair may be further conditioned and/or rinsed after shampooing and before application of the compositions disclosed herein. After the hair is shampooed, conditioned, and/or rinsed, the hair color altering composition is then applied to the washed or rinsed hair when the hair is wet, damp, or moist. In some other embodiments, before applying the hair color altering composition to the hair, the hair can optionally be moistened, damped, or wetted by water spray or using a wet towel, or by applying other treatment compositions that make the hair moist, damp, or wet. In some embodiments, after the application of the compositions disclosed to the hair, the hair may be further rinsed, shampooed, and/or conditioned one or more times.

As used herein, the term “effective amount” refers to any amount sufficient to provide a desired lifting or coloring effect. For example, in some embodiments, from about 0.1 grams to about 50 grams of a hair color altering composition may be applied to the hair, depending on the specific product formulation, hair length, hair volume, and hair color. In some embodiments, about 0.1 grams of the composition per gram of hair is applied to the hair. In some embodiments, about 0.5 grams of the composition per gram of hair is applied to the hair. The composition applied to the hair may be distributed through the hair as desired, e.g. substantially evenly or uniformly massaged throughout the hair by combing through with fingers or a means such as a comb or the like.

After the hair color altering composition is applied on to the hair, the composition may be allowed to remain on the hair for a period of time sufficient to impart the benefits provided by the composition (“leave-in” or “processing” time), such as lifting effect or providing desired color or tone to the hair. For example, the hair color composition may be left on the hair for up to one hour, such as from about 1 minute to about 60 minutes, from about 1 minutes to about 45 minutes, from about 1 minute to about 30 minutes, from about 1 minute to about 20 minutes, from about 1 minute to about 15 minutes, from about 1 minute to about 10 minutes, from about 1 minute to about 5 minutes, from about 3 minutes to about 45 minutes, from about 5 minutes to about 30 minutes, or from about 10 minutes to about 20 minutes, and then removed, e.g. rinsed off, from the hair. One skilled in the art will, by considering various factors such as the starting color of the hair and the desired color to obtained, be able to determine an appropriate amount of time to leave the hair color altering composition or mixture on the hair in order to achieve the desired alternation in hair color. If desired, the hair treated with hair color altering compositions may be further shampooed and/or conditioned after the mixture is rinsed off the hair.

In some embodiments, a method disclosed herein is for permanently or demi-permanently coloring or dyeing hair, while improving the strength, elasticity, and/or fiber integrity of the hair. In this case, a hair color base composition may include one or more coloring agents such as oxidation dyes optionally combined with one or more couplers.

Having described the many embodiments of the present invention in detail, it will be apparent that modifications and variations are possible without departing from the scope of the disclosure defined in the appended claims. Furthermore, it should be appreciated that all examples in the present disclosure, while illustrating many embodiments of the disclosure, are provided as non-limiting examples and are, therefore, not to be taken as limiting the various aspects so illustrated. It is to be understood that all definitions herein are provided for the present disclosure only.

As used herein, the terms “comprising,” “having,” and “including” (or “comprise,” “have,” and “include”) are used in their open, non-limiting sense. The phrase “consisting essentially of” limits the scope of a claim to the specified materials or steps and those that do not materially affect the basic and novel characteristics of the compositions.

In this application, the use of the singular includes the plural unless specifically stated otherwise. The singular forms “a,” “an,” “the,” and “at least one” are understood to encompass the plural as well as the singular unless the context clearly dictates otherwise. The expression “one or more” means “at least one” and thus includes individual components as well as mixtures/combinations. Likewise, the term “a salt thereof” also relates to “salts thereof.” Thus, where the disclosure refers to “an element selected from the group consisting of A, B, C, D, E, F, a salt thereof, or mixtures thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

The term “and/or” should be understood to include both the conjunctive and the disjunctive. For example, “citric acid and/or salts thereof” means “citric acid and salts thereof” as well as “citric acid or salts thereof.”

As used herein, the phrases “and mixtures thereof,” “and a mixture thereof,” “and combinations thereof,” “and a combination thereof,” “or mixtures thereof,” “or a mixture thereof,” “or combinations thereof,” and “or a combination thereof,” are used interchangeably to denote that the listing of components immediately preceding the phrase, such as “A, B, C, D, or mixtures thereof” signify that the component(s) may be chosen from A, from B, from C, from D, from A+B, from A+B+C, from A+D, from A+C+D, etc., without limitation on the variations thereof. Thus, the components may be used individually or in any combination thereof.

For purposes of the present disclosure, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

All ranges and amounts given herein are intended to include sub-ranges and amounts using any disclosed point as an end point, and all endpoints are intended to be included unless expressly stated otherwise. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not expressly stated, unless expressly stated otherwise. Similarly, a range given of “about 1% to 10%” is intended to have the term “about” modifying both the 1% and the 10% endpoints. The term “about” is used herein to indicate a difference of up to +/−10% from the stated number, such as +/−9%, +/−8%, +/−7%, +/−6%, +/−5%, +/−4%, +/−3%, +/−2%, or +/−1%. Unless expressly stated otherwise, “about” means+/−5%. Likewise, all endpoints of ranges are understood to be individually disclosed, such that, for example, a range of 1:2 to 2:1 is understood to disclose a ratio of both 1:2 and 2:1.

As used herein, if a component is described as being present “in an amount up to” a certain amount, it is intended that such component is, in fact, present in the composition, i.e. is present in an amount greater than 0%.

All amounts given herein are relative to the amount of active material, unless otherwise indicated.

All percentages, parts and ratios herein are based upon the total weight of the compositions of the present disclosure, unless otherwise indicated.

The term “altering the color” or “color altering” as used herein refers to lifting or lightening the color of hair. It can also refer to dyeing or coloring hair or depositing color onto the hair. In certain instances, it refers to lifting or lightening the color of hair and depositing color onto the hair at the same time.

As used herein, the terms “applying a composition onto keratin fibers” and “applying a composition onto hair” and variations of these phrases are intended to mean contacting the keratin fibers including hair, with at least one of the compositions of the disclosure, in any manner. It may also mean contacting the keratin fibers or hair in an effective amount of the composition.

As used herein, the term “salts” may include salts having a counter-ion such as an alkali metal, alkaline earth metal, or ammonium counterion. This list of counterions, however, is non-limiting. Salts also include a dissociated form of a compound, e.g. in an aqueous solution.

As used herein, the term “stable” means that the composition does not exhibit phase separation for a period of time, for example, for at least 1 day (24 hours), one week, one month, one year, or two years.

As used herein, the terms “substantially free” or “essentially free” mean the specific material may be present in small amounts that do not materially affect the basic and novel characteristics of the compositions according to the disclosure. For instance, there may be less than 2% by weight of a specific material added to a composition, based on the total weight of the compositions (provided that an amount of less than 2% by weight does not materially affect the basic and novel characteristics of the compositions according to the disclosure. Similarly, the compositions may include less than 2%, less than 1.5%, less than 1%, less than 0.5%, less than 0.1%, less than 0.05%, or less than 0.01%, or none of the specified material. Furthermore, all components that are positively set forth in the instant disclosure may be negatively excluded from the claims, e.g., a claimed composition may be “free,” “essentially free” (or “substantially free”) of one or more components that are positively set forth in the instant disclosure. The terms “free,” “substantially free,” and “essentially free” as used herein mean that the specific material is not added to the composition but may still be present in a raw material that is included in the composition. For example, a composition according to the disclosure may not include an added wax but may include a pigment that is coated with a wax and still be considered “free,” “substantially free,” or “essentially free” of waxes.

As used herein, the term “surfactants,” as well as any specifically identified surfactants, includes salts of the surfactants even if not explicitly stated.

As used herein, the term “treat” (and its grammatical variations) refers to the application of the compositions of the present disclosure onto the surface of keratin materials, such as hair.

Unless otherwise expressly stated, it is in no way intended that any method set forth herein be construed as requiring that its steps be performed in a specific order. Accordingly, where a method claim does not expressly recite an order to be followed by its steps or it is not specifically stated in the claims or descriptions that the steps are to be limited to a specific order, it is no way intended that any particular order be inferred.

### EXAMPLES

The following examples are intended to be non-limiting and explanatory in nature only. In the Examples, amounts in each composition are expressed in percentage by weight (wt %) of active materials, unless otherwise defined, relative to the total weight of the composition.

### Example 1-Hair Color Base Compositions

Exemplary hair color base compositions 1A-1D shown in Table 1A were prepared.

TABLE 1A
Exemplary Hair Base Compositions

| | 1A | 1B | 1C | 1D |
|---|---|---|---|---|
| MICA AND/OR | 0.85 | 0.50 | 0.49 | 0.50 |
| TITANIUM DIOXIDE | | | | |
| AMMONIUM HYDROXIDE | 4.12 | | 4.12 | |
| ETHANOLAMINE | 1.00 | 5.24 | 1.00 | 5.27 |
| POLYQUATERNIUM-22 | 0.82 | 0.82 | 0.82 | 0.82 |
| GLYCINE | 0.50 | 0.50 | 0.50 | 0.50 |
| CITRIC ACID | 0.20 | 0.20 | 0.20 | 0.20 |
| GLYCOL DISTEARATE | 3.00 | 2.25 | 3.33 | 2.25 |
| DIMETHICONE | | | 0.01 | |
| CETEARYL ALCOHOL | 11.00 | 7.00 | 11.00 | 7.00 |
| OLEYL ALCOHOL | 3.00 | 2.10 | 3.60 | 2.10 |
| HYDROGENATED | 2.00 | 1.50 | 2.00 | 1.50 |
| VEGETABLE OIL | | | | |
| OLEIC ACID | 3.00 | 2.10 | 3.00 | 2.10 |
| LAURETH-12 AND OLETH-30 | 7.00 | 7.00 | 7.00 | 7.00 |
| HYDROXYETHYLCELLULOSE | 0.20 | 0.40 | 0.20 | 0.40 |
| CETYL | 0.09 | 0.18 | 0.09 | 0.18 |
| HYDROXYETHYLCELLULOSE | | | | |
| HAIR COLOR AGENT(S) | QS | QS | QS | QS |
| ADDITIVES (fragrance, fillers, | ≤3 | ≤3 | ≤3 | ≤3 |
| preservatives, buffers, chelants) | | | | |
| SOLVENTS (water and | QS | QS | QS | QS |
| non-aqueous solvents) | to 100 | to 100 | to 100 | to 100 |

Compositions 1A-1D are hair color base compositions according to the disclosure to which any combination of hair color agents as described herein can be added, if desired. For example, one or more oxidative dye compounds such as m-aminophenol, p-aminophenol, 2-methyl-5-hydroxyethylaminophenol, 2,4-diaminophenoxyethanol hydrochloride, 4-amino-2-hydroxytoluene, 6-hydroxyindole, etc., and/or one or more direct dyes such as HC Red No. 3, Basic Red 5, Basic Yellow 87, Basic Orange 31, 2-Amino-6-Chloro-4-Nitrophenol, 3-Nitro-p-Hydroxyethylaminophenol, 2-Nitro-5-Glyceryl, etc., can be included as described above.

Comparative hair color base compositions C1-C5 shown in Table 1B were prepared.

TABLE 1B
Comparative Hair Base Compositions

| | C1 | C2 | C3 | C4 | C5 |
|---|---|---|---|---|---|
| MICA AND/OR TITANIUM DIOXIDE | 0.49 | 0.15 | 0.15 | 0.15 | 0.50 |
| AMMONIUM HYDROXIDE | 4.12 | 4.12 | 4.12 | 4.12 | |
| ETHANOLAMINE | 1.00 | 1.20 | 4.20 | 1.80 | 5.24 |
| POLYQUATERNIUM-6 | | 0.40 | | | |
| POLYQUATERNIUM-22 | 0.82 | | | | 0.82 |
| TAURINE | | | | 0.50 | |
| CITRIC ACID | | | 1.00 | 0.20 | |
| GLYCOL DISTEARATE | 3.00 | 3.00 | 3.00 | 3.00 | 2.25 |
| DIMETHICONE | 0.01 | 0.03 | 0.03 | 0.03 | |
| CETEARYL ALCOHOL | 11.00 | 14.00 | 14.00 | 14.00 | 7.00 |
| OLEYL ALCOHOL | 3.60 | 1.80 | 1.80 | 1.80 | 2.10 |
| HYDROGENATED VEGETABLE OIL | 2.00 | 2.00 | 2.00 | 2.00 | 1.50 |
| OLEIC ACID | 3.00 | 2.00 | 2.00 | 2.00 | 2.10 |
| LAURETH-12 AND OLETH-30 | 7.00 | 7.00 | 7.00 | 7.00 | 7.00 |
| HYDROXYETHYLCELLULOSE | 0.20 | 0.20 | 0.20 | 0.20 | 0.40 |
| CETYL HYDROXYETHYLCELLULOSE | 0.09 | 0.09 | 0.09 | 0.09 | 0.18 |
| HAIR COLOR AGENT(S) | QS | QS | QS | QS | QS |
| ADDITIVES (fragrance, fillers, | ≤3 | ≤3 | ≤3 | ≤3 | ≤3 |
| preservatives, buffers, chelants) | | | | | |
| SOLVENTS (water and | QS | QS | QS | QS | QS |
| non-aqueous solvents) | to 100 | to 100 | to 100 | to 100 | to 100 |

The pH of each of compositions 1A-1D and C1-C5 was in the range of about 10-11.

### Example 2—Developer Compositions

An exemplary 20V developer composition D1 and an exemplary 10V developer composition D2 are shown in Table 2. The developers had a pH of about 2.2.

TABLE 2
Developer Compositions

| | D1 | D2 |
|---|---|---|
| TRIDECETH-2 CARBOXAMIDE MEA | 0.85 | 0.82 |
| TETRASODIUM ETIDRONATE | 0.06 | 0.06 |
| SODIUM SALICYLATE | 0.035 | 0.035 |
| GLYCERIN | 0.50 | 0.50 |
| CETEARYL ALCOHOL | 2.28 | 2.28 |
| CETEARETH-25 | 0.57 | 0.57 |
| HYDROGEN PEROXIDE | 6.0 | 3.0 |
| TETRASODIUM PYROPHOSPHATE | 0.04 | 0.04 |
| WATER | QS to 100 | QS to 100 |

### Example 3—Evaluation of Fiber Integrity of Treated Hair

The fiber integrity of hair treated with compositions according to the disclosure or comparative compositions was evaluated.

### Example 3A—Evaluation of Permanent Hair Color Altering Compositions

Caucasian hair tresses (4) with tone level 4-5 and weighing 2.7 grams were used. The hair tresses were pre-bleached with a mixture of a commercially-available bleaching composition and developer. The bleaching mixture was left on the hair to process for 50 minutes at 33° C. After processing, the hair tresses were rinsed and washed with a commercially-available shampoo, rinsed again, and then blow-dried using a hair dryer for 2 minutes.

Just before use, each of hair color base compositions 1C and C1 was mixed with developer D1 in a mix ratio of 1:1 to prepare permanent hair color altering compositions 1C+D1 or C1+D1. The following protocol was then performed four times for each tress.

First, each of the permanent hair color altering compositions was applied to two separate pre-bleached hair tresses (10 grams of mixture per tress), left on the hair to process for 35 minutes at 27° C., and then rinsed from the hair. Next, the hair was subjected to a shampoo cycle that consisted of shampooing the hair with a commercially-available shampoo, rinsing the hair, and then drying the hair using a blow dryer for 2 minutes. The shampoo cycle was repeated six times, for a total of seven shampoo cycles. Finally, once the protocol was performed a total of four times for each hair tress, the tresses were stored overnight at room temperature and 60% relative humidity.

Break stress of the treated hair tresses (wet, 50 fibers from each tress) was evaluated using a Dia-Stron Mini Tensile Tester (MTT). Break stress measures the force/area needed to break the hair fiber. The higher the break stress, the stronger the fiber. The break stress value of virgin hair (no prior chemical treatments) of the type used in Example 3A has been found to be about 136 MPa.

The results, shown in Table 3A, were considered to be statistically significant. JMP™ analytical software was used to calculate the statistics (student's t-test at 95% confidence level).

TABLE 3A
Break Stress

| | | | Standard Error |
|---|---|---|---|
| Treatment | Mean (MPa) | Standard Deviation | Mean |
| 1C + D1 | 135 | 19.5 | 2.8 |
| C1 + D1 | 122 | 34.1 | 4.8 |

As shown in Table 3A, the process of altering the color of the pre-bleached hair using the comparative hair color altering composition (C1+D1) resulted in hair that had a statistically significant decreased break stress value, indicating that the hair was not as strong as, and more damaged than, the hair treated with hair color altering composition 1C+D1, which included glycine and citric acid (p<0.05). Surprisingly, the break stress of the hair that was bleached and then treated with hair color altering composition 1C+D1 was almost the same as that of virgin hair. These results demonstrate that hair treated with compositions according to the disclosure had greater fiber integrity and strength even after being subjected to a chemical hair color altering process, compared to hair subjected to the same process but without the beneficial agents according to the disclosure.

### Example 3B—Evaluation of Comparative Permanent Hair Color Altering Compositions

In order to compare the fiber integrity benefits imparted to hair by the combination of glycine and carboxylic acids with those imparted by compositions containing other amino acids and carboxylic acids, containing carboxylic acids but no amino acids, or containing neither carboxylic acids or amino acids, the procedure of Example 3A was repeated with comparative hair color base compositions C1-C4. The break stress results are shown in Table 3B.

TABLE 3B
Break Stress

| | | | Standard Error |
|---|---|---|---|
| Treatment | Mean (MPa) | Standard Deviation | Mean |
| C1 + D1 | 128 | 25.2 | 4.3 |
| C2 + D1 | 123 | 26.0 | 3.7 |
| C3 + D1 | 115 | 33.6 | 4.9 |
| C4 + D1 | 126 | 30.5 | 3.6 |

The results in Table 3B show that the strength of hair treated with comparative hair color altering compositions C1+D1 and C2+D1 (no amino acids and no carboxylic acids) was similar to that of comparative hair color altering composition C4+D1, having a combination of taurine and citric acid, and all the strength of all three was greater than that of hair treated with comparative hair color altering composition C3+D1, having citric acid but no amino acids. However, none of the results in Table 3B show break stress values as high as that seen in Table 3A for hair treated with hair color altering composition 1C+D1.

The data in Tables 3A and 3B therefore confirm that the combination of glycine with carboxylic acids provides a surprisingly and statistically significant improvement in fiber strength of hair treated in harsh chemical processes such as bleaching and dyeing.

### Example 3C—Evaluation of Demi-Permanent Hair Color Altering Compositions

In order to evaluate fiber integrity benefits imparted by demi-permanent hair color altering compositions according to the disclosure, additional comparative tests were performed.

Caucasian hair tresses (5) with tone level 4 were pre-bleached with a mixture of a commercially-available bleaching composition and a 40V hydrogen peroxide developer. The bleaching mixture was left on the hair to process for 50 minutes at 33° C., in foils. After processing, the hair tresses were rinsed and washed with a commercially-available shampoo, rinsed again, and then blow-dried. The bleaching protocol was repeated one additional time. These bleaching conditions were considered to be harsh, and would be expected to cause significant damage to the hair.

Just before use, each of hair color base compositions 1B and C5 was mixed with developer D2 in a mix ratio of 1:1 to prepare demi-permanent hair color altering compositions 1B+D2 or C5+D2. Next, each of the demi-permanent hair color altering compositions was applied to two separate pre-bleached, damp hair tresses (10 grams of mixture per tress), left on the hair to process for 10 minutes at 27° C., and then rinsed from the hair. Finally, the hair was shampooed with a commercially-available shampoo and rinsed. The testing had one bleached tress that was not treated with a hair color altering composition as a control (CNT).

The break stress of the tresses (wet) was then evaluated, as described in Example 3A. The results are shown in Table 3C.

TABLE 3C
Break Stress

| Treatment | Mean (MPa) | Standard Deviation |
|---|---|---|
| 1B + D2 | 133 | 13.3 |
| C5 + D2 | 117 | 20.5 |
| CNT1 | 113 | 15.2 |

The break stress data in Table 3C confirm the surprising strength benefits imparted to the hair with the combination of glycine and carboxylic acids, such as citric acid. The results, which are also statistically significant, are particularly surprising considering the severity of the bleaching process, and the break stress value of the control tress confirms this.

In addition, the elastic modulus of the tresses (wet) was evaluated, using the same MTT instrument. Measuring elastic modulus provides information about the elasticity of the hair, where hair that is more elastic (higher elastic modulus) is healthier and more likely to return to its natural state if stretched. The results are shown in Table 3D.

TABLE 3D
Elastic Modulus

| Treatment | Mean (MPa) | Standard Deviation |
|---|---|---|
| 1B + D2 | 871 | 196 |
| C5 + D2 | 759 | 196 |
| CNT1 | 723 | 216 |

The elastic modulus data in Table 3D further demonstrate that hair treated with hair color altering compositions according to the disclosure provide fiber integrity benefits to hair in processes for altering the color of hair. The difference in the measured elastic modulus values for the hair treated with hair color altering composition 1B+D2 is surprisingly greater than the hair treated with comparative hair color altering composition C5+D2 or the control, and this difference is considered to be statistically significant.

### Example 4—Additional Compositions

The following additional hair color base compositions according to the disclosure (2A-2F) can be prepared and combined at or near the time of use with a developer composition to form a hair color altering composition according to the disclosure that is expected to provide hair fiber integrity results similar to those observed with compositions 1B and 1C in Example 3.

TABLE 4

| | 2A | 2B | 2C | 2D |
|---|---|---|---|---|
| GLYCINE | 0.50 | 0.50 | 0.10 | 1.50 |
| CITRIC ACID | 0.20 | | 0.025 | 1.00 |
| LACTIC ACID and/or MALIC | | 0.20 | | |
| ACID | | | | |
| AMMONIUM HYDROXIDE | 4.12 | 4.12 | 4.12 | 4.12 |
| CETEARYL ALCOHOL | 14.00 | 14.00 | 14.00 | 14.00 |
| CETYL | 0.09 | 0.09 | 0.09 | 0.09 |
| HYDROXYETHYLCELLULOSE | | | | |
| ETHANOLAMINE | 1.80 | 1.80 | 1.80 | 1.80 |
| GLYCOL DISTEARATE | 3.00 | 3.00 | 3.00 | 3.00 |
| HYDROGENATED | 2.00 | 2.00 | 2.00 | 2.00 |
| VEGETABLE OIL | | | | |
| HYDROXYETHYLCELLULOSE | 0.20 | 0.20 | 0.20 | 0.20 |
| OLEIC ACID | 2.00 | 2.00 | 2.00 | 2.00 |
| LAURETH-12 AND OLETH-30 | 7.00 | 7.00 | 7.00 | 7.00 |
| OLEYL ALCOHOL | 1.80 | 1.80 | 1.80 | 1.80 |
| TITANIUM DIOXIDE | 0.15 | 0.15 | 0.15 | 0.15 |
| HAIR COLOR AGENT(S) | QS | QS | QS | QS |
| ADDITIVES (fragrance, fillers, | ≤3 | ≤3 | ≤3 | ≤3 |
| preservatives, buffers, chelants) | | | | |
| SOLVENTS (water and | QS | QS | QS | QS |
| non-aqueous solvents) | to 100 | to 100 | to 100 | to 100 |

### Example 5—Additional Compositions

The following additional hair color base compositions according to the disclosure (3A-3B) and comparative compositions (C6-C7) were prepared.

TABLE 5

| | 3A | 3B | C6 | C7 |
|---|---|---|---|---|
| TAURINE | | | 1.00 | 0.25 |
| GLYCINE | 1.00 | 0.25 | | |
| CITRIC ACID | 0.40 | 0.10 | 0.40 | |
| LACTIC ACID | | | | 0.10 |
| CETEARYL ALCOHOL | 7.00 | 7.00 | 7.00 | 7.00 |
| CETYL HYDROXYETHYLCELLULOSE | 0.18 | 0.18 | 0.18 | 0.18 |
| ETHANOLAMINE | 5.62 | 5.62 | 5.62 | 5.62 |
| GLYCOL DISTEARATE | 2.25 | 2.25 | 2.25 | 2.25 |
| HYDROGENATED VEGETABLE OIL | 1.50 | 1.50 | 1.50 | 1.50 |
| HYDROXYETHYLCELLULOSE | 0.40 | 0.40 | 0.40 | 0.40 |
| OLEIC ACID | 2.10 | 2.10 | 2.10 | 2.10 |
| LAURETH-12 AND OLETH-30 | 7.00 | 7.00 | 7.00 | 7.00 |
| OLEYL ALCOHOL | 2.10 | 2.10 | 2.10 | 2.10 |
| TITANIUM DIOXIDE | 0.15 | 0.15 | 0.15 | 0.15 |
| TOLUENE-2,5-DIAMINE (and) | 0.365 | 0.365 | 0.365 | 0.365 |
| THIOGLYCERIN | | | | |
| m-AMINOPHENOL | 0.0799 | 0.0799 | 0.0799 | 0.0799 |
| HYDROXYBENZOMORPHOLINE | 0.1756 | 0.1756 | 0.1756 | 0.1756 |
| 2,4-DIAMINOPHENOXYETHANOL HCL | 0.0143 | 0.0143 | 0.0143 | 0.0143 |
| 6-HYDROXYINDOLE | 0.0252 | 0.0252 | 0.0252 | 0.0252 |
| N,N-BIS(2-HYDROXYETHYL)-p- | 0.0316 | 0.0316 | 0.0316 | 0.0316 |
| PHENYLENEDIAMINE SULFATE | | | | |
| 2-AMINO-3-HYDROXYPYRIDINE | 0.0191 | 0.0191 | 0.0191 | 0.0191 |
| HYDROXYETHYL-3,4- | 0.1781 | 0.1781 | 0.1781 | 0.1781 |
| METHYLENEDIOXYANILINE HCL | | | | |
| ADDITIVES (fragrance, fillers, | ≤3 | ≤3 | ≤3 | ≤3 |
| preservatives, buffers, chelants) | | | | |
| SOLVENTS (water and non-aqueous | Q.S. | Q.S. | Q.S. | Q.S. |
| solvents) | | | | |
| pH | 10.20 | 10.25 | 10.27 | 10.25 |
| VISCOSITY (cP) | 4944 | 6093 | 5319 | 6445 |

Upon preparation, each of compositions 3A-3B and C6-C7 had a homogenous, cream-like texture. The pH values and viscosities of compositions 3A-3B and C6-C7 were measured upon preparation. The viscosity was measured at 25° C., 200 RPM for 60 seconds using an M4 spindle (Rheomat 180). After 24 hours, the compositions all still had a homogenous, cream-like texture.

Hair color base compositions 3A-3B can be combined at or near the time of use with a developer composition to form a hair color altering composition according to the disclosure that is expected to provide hair fiber integrity results similar to those observed with compositions 1B and 1C in Example 3. Comparative hair color base compositions C6-C7 can be combined at or near the time of use with a developer composition to form a comparative hair color altering composition that is expected to provide hair fiber integrity results similar to those observed with composition C4 in Example 3.

1. A hair color base composition comprising:
(a) from about 0.03% to about 1% of citric acid and/or a salt thereof;
(b) from about 0.1% to about 1.5% of glycine and/or a salt thereof;
(c) a solvent comprising water and optionally one or more non-aqueous solvents;
(d) one or more oxidative dye compounds;
(e) one or more colloid agents; and
(f) one or more alkalizing agents,
wherein all amounts are by weight, relative to the total weight of the composition.

2. The hair color base composition according to claim 1, wherein the total amount of (a) citric acid and salts thereof ranges from about 0.05% to about 0.5% by weight, relative to the total weight of the composition.

3. The hair color base composition according to claim 1, wherein the total amount of (b) glycine and salts thereof ranges from about 0.2% to about 1.1% by weight, relative to the total weight of the composition.

4. The hair color base composition according to claim 1, wherein the composition has a weight ratio of the total amount of (b) glycine and salts thereof to the total amount of (a) citric acid and salts thereof that is greater than about 1.

5. The hair color base composition according to claim 1, wherein the total amount of colloid agents ranges from about 0.01% to about 2% by weight, relative to the total weight of the composition.

6. The hair color base composition according to claim 1, further comprising (g) one or more fatty alcohols, wherein the total amount of fatty alcohols ranges from about 1% to about 25% by weight, relative to the total weight of the composition.

7. The hair color base composition according to claim 1, further comprising at least one additional component chosen from surfactants, cationic polymers, fatty compounds other than fatty alcohols, or combinations of two or more thereof.

8. The hair color base composition according to claim 1, wherein the (f) alkalizing agent comprises monoethanolamine.

9. The hair color base composition according to claim 1, wherein the (e) colloid agent comprises cellulose and/or a derivative thereof.

10. The hair color base composition according to claim 1, comprising:
(a) from about 0.03% to about 1% of citric acid and/or a salt thereof;
(b) from about 0.1% to about 1.5% of glycine and/or a salt thereof;
(c) water;
(d) one or more oxidative dye compounds;
(e) from about 0.01% to about 2% of cellulose and/or a derivative thereof;
(f) from about 0.1% to about 10% of one or more alkalizing agents; and
(g) from about 2% to about 20% of one or more fatty alcohols,
wherein all amounts are by weight, relative to the total weight of the composition.

11. A method of altering the color of hair comprising:
(i) preparing a hair color altering composition by mixing an oxidizing composition comprising one or more oxidizing agents with a hair color base composition comprising:
(a) from about 0.03% to about 1% of citric acid and/or a salt thereof;
(b) from about 0.1% to about 1.5% of glycine and/or a salt thereof;
(c) a solvent comprising water and optionally one or more non-aqueous solvents;
(d) one or more oxidative dye compounds;
(e) one or more colloid agents; and
(f) one or more alkalizing agents,
wherein all amounts are by weight, relative to the total weight of the composition, and
(ii) applying the hair color altering composition to the hair.

12. The method according to claim 11, wherein the total amount of (a) citric acid and salts thereof in the hair color base composition ranges from about 0.05% to about 0.5% by weight, relative to the total weight of the hair color base composition.

13. The method according to claim 11, wherein the total amount of (b) glycine and salts thereof in the hair color base composition ranges from about 0.2% to about 1.1% by weight, relative to the total weight of the hair color base composition.

14. The method according to claim 11, wherein the hair color base composition has a weight ratio of the total amount of (b) glycine and salts thereof to the total amount of (a) citric acid and salts thereof that is greater than about 1.

15. The method according to claim 11, wherein the total amount of colloid agents in the hair color base composition ranges from about 0.01% to about 2% by weight, relative to the total weight of the hair color base composition.

16. The method according to claim 11, wherein the hair color base composition further comprises (g) one or more fatty alcohols, wherein the total amount of fatty alcohols ranges from about 1% to about 25% by weight, relative to the total weight of the hair color base composition.

17. The method according to claim 11, wherein the hair color base composition further comprises at least one additional component chosen from surfactants, cationic polymers, fatty compounds other than fatty alcohols, or combinations of two or more thereof.

18. The method according to claim 11, wherein the (f) alkalizing agent in the hair color base composition comprises monoethanolamine.

19. The method according to claim 11, wherein the (e) colloid agent in the hair color base composition comprises cellulose and/or a derivative thereof.

20. The method according to claim 11, further comprising (iii) rinsing the hair color altering composition from the hair after a leave-in period of time ranging from about 1 minute to about 60 minutes.
